(19) United States
(12) Patent Application Publication
(10) Pub. No.: US 2019/0177430 A1
(43) Pub. Date: Jun. 13, 2019

(54) Methods of Treating Cancers With Chemotherapy With Reduced Toxicity

(71) Applicants: The Wistar Institute of Anatomy and Biology, Philadelphia, PA (US); The Trustees of The University of Pennsylvania, Philadelphia, PA (US)

(72) Inventors: Bonnie Ky, Philadelphia, PA (US); David W. Speicher, Berwyn, PA (US)

(21) Appl. No.: 16/327,654

(22) PCT Filed: Aug. 24, 2017
(86) PCT No.: PCT/US2017/048415

§ 371 Date: Feb. 22, 2019

Related U.S. Application Data
(60) Provisional application No. 62/378,949, filed on Aug. 24, 2016.

(51) Int. Cl. C07K 16/32; A61P 35/00; G01N 33/574; G01N 33/50

(52) U.S. Cl. CPC C07K 16/32; A61P 35/00; G01N 33/57415; A61K 31/704; C07K 2317/24; C07K 2317/76; G01N 33/5014

(57) ABSTRACT

In some embodiments, therapeutic treatments for a disease such as a cancer are disclosed, including pharmaceutical compositions and methods of using pharmaceutical compositions for treating a cancer in a human subject wherein the human subject exhibits an elevated concentration of immunoglobulin E (IgE) in plasma obtained from the human subject, comprising the step of administering a therapeutically effective amount of a chemotherapeutic regimen to the human subject in need thereof. In some embodiments, the chemotherapeutic regimen includes doxorubicin monotherapy, trastuzumab monotherapy, doxorubicin and trastuzumab combination therapy, doxorubicin, cyclophosphamide, and 5-fluorouracil combination therapy, and doxorubicin, cyclophosphamide, paclitaxel, and trastuzumab combination therapy.

### CROSS-REFERENCE TO RELATED APPLICATIONS

This international application claims the benefit of U.S. Provisional Application No. 62/378,949, filed Aug. 24, 2016, the entirety of which is incorporated herein by reference.

### GOVERNMENT SUPPORT

This invention was made with government support under Grant Nos. CA010815, HL118018, and HL095661 awarded by the National Institutes of Health. The government has certain rights in the invention.

### FIELD OF THE INVENTION

Therapeutic treatments of cancers using methods to reduce toxicity, including cardiotoxicity, are disclosed.

### BACKGROUND OF THE INVENTION

Doxorubicin and trastuzumab (Herceptin®) are used widely in the treatment of breast cancer, are highly effective, and have led to important survival gains. Slamon et al., New Engl. J. Med. 2001, 344, 783-792. However, these agents carry a substantially increased risk of cardiovascular morbidity and mortality. Doxorubicin-induced cardiac dysfunction occurs in 9% of treated patients at dosages of 250 mg/m². Swain et al., Cancer 2003, 97, 2869-2879. Trastuzumab, a highly effective humanized monoclonal antibody used in the treatment of Her2 (ErbB2)-positive breast cancer, also causes significant declines in left ventricular ejection fraction (EF), resulting in potential delays or cessation of necessary therapy. Suter et al., J. Clin. Oncol. 2007, 25, 3859-3865; Tan-Chiu et al., J. Clin. Oncol. 2005, 23, 7811-7819. When anthracyclines and trastuzumab are used in combination, up to 18% of patients develop cancer therapeutics-related cardiac dysfunction (CTRCD) and 2-4% of patients develop severe, symptomatic heart failure. Telli et al., J. Clin. Oncol. 2007, 25, 3525-3533. Trastuzumab is a monoclonal antibody that binds to HER2 (also known as HER2 (ErbB2 or p185^{neu})).

There is an important need to identify patients at increased risk of developing CTRCD, particularly prior to the development of overt disease. Cardiovascular biomarkers have been widely studied as potential tools to risk stratify patients; however, many of these markers are neither specific nor sensitive for diagnosing CTRCD or predicting which patients are at increased cardiovascular risk. Moreover, the lack of mechanistic understanding of the underlying pathophysiology of doxorubicin and trastuzumab cardiac dysfunction has hindered the development of newer markers. As such, discovery proteomics represents a unique opportunity to identify novel pathways and biomarkers.

Traditionally, there have been a number of hurdles that have hindered advances in proteomics discovery. These include the great complexity of the plasma or serum proteome; the presence of a few very high-abundant proteins; the wide dynamic range of protein concentrations; and the molecular heterogeneity of many proteins. However, considerable advances have been made in liquid chromatography-mass spectrometry (LC-MS) based biomarker discovery and validation methods over the past several years that have substantially reduced these barriers. Now, rigorous and powerful proteomic-based technologies can be used to successfully discover and validate new biomarkers.

The invention provides the unexpected finding that human subjects with cancer, including breast cancer, that also exhibit high concentrations of immunoglobulin E (IgE), as described herein, represent a selected subclass of subjects with a unique disease that may be treated using chemotherapy regimens such that cardiac toxicity is avoided.

### SUMMARY OF THE INVENTION

The invention described herein includes methods of treating cancer in a human subject and/or preventing injury in a human subject being treated for cancer. The invention described herein also includes kits for practicing such methods.

In an embodiment, the invention includes a method of treating a cancer in a human subject wherein the human subject exhibits an elevated concentration of immunoglobulin E (IgG) in plasma obtained from the human subject. In some embodiments, the human subject may exhibit an elevated concentration of IgE in plasma obtained from the subject prior to the initiation of treatment and/or the provision of a chemotherapy regimen. In some embodiments, the method may include the step of administering a therapeutically effective amount of a chemotherapeutic regimen to the human subject in need thereof.

In some embodiments, the chemotherapeutic regimen may include one or more of a doxorubicin monotherapy; trastuzumab monotherapy; doxorubicin and trastuzumab combination therapy; doxorubicin, cyclophosphamide, and 5-fluorouracil combination therapy; and doxorubicin, cyclophosphamide, paclitaxel, and trastuzumab combination therapy.

In some embodiments, the cancer may be breast cancer.

In some embodiments, the human subject may exhibit a reduced concentration of beta-hydroxylase in plasma obtained from the human subject.

In some embodiments, the human subject may exhibit a reduced concentration of cathepsin S in plasma obtained from the human subject.

In some embodiments, the elevated concentration of IgE may be determined by an IgE-specific protein assay. In certain embodiments, the IgE-specific protein assay may be an enzyme-linked immunosorbent assay (ELISA). In certain embodiments, the IgE-specific protein assay may be a liquid chromatography mass spectrometry (LC-MS) assay.

In some embodiments, the elevated concentration of IgE may be determined as a measurement of IgE concentration in plasma, wherein the IgE concentration in plasma may be selected from the group consisting of greater than 150 ng/mL, greater than 200 ng/mL, greater than 300 ng/mL, and greater than 400 ng/mL. In certain embodiments, the IgE concentration in plasma may be about 188 ng/mL.

In some embodiments, the elevated concentration of IgE may be determined as a measurement of IgE relative to immunoglobulin G1 (IgG1) in plasma (IgE/IgG1 ratio), wherein IgE/IgG1 ratio may be selected from the group consisting of greater than 1.5×10⁻⁵, greater than 2×10⁻⁵, greater than 2.5×10⁻⁵, greater than 3×10⁻⁵, greater than 4×10⁻⁵, and greater than 5×10⁻⁵. In certain embodiments, the IgE/IgG1 ratio may be about 2.1×10⁻⁵.

In another embodiment, the invention includes a method of treating a cancer in a human subject that includes the step of obtaining a plasma sample from the human subject. The method may include the step of analyzing the plasma sample by an immunoglobulin E (IgE)-specific protein assay for IgE. The method may include the step of determining whether the human subject is at a low risk for cardiac injury from a chemotherapeutic regimen based on an elevated plasma IgE concentration. The method may include the step of administering a chemotherapeutic regimen to the human subject determined to have the low risk of cardiac injury.

In some embodiments, the chemotherapeutic regimen may include one or more of doxorubicin monotherapy; trastuzumab monotherapy; doxorubicin and trastuzumab combination therapy; doxorubicin, cyclophosphamide, and 5-fluorouracil combination therapy; and doxorubicin, cyclophosphamide, paclitaxel, and trastuzumab combination therapy.

In some embodiments, the cancer may be breast cancer.

In some embodiments, the method may include one or more of the steps of analyzing the plasma sample for beta-hydroxylase and determining the risk of cardiac injury in the human subject based on reduced beta-hydroxylase concentration.

In some embodiments, the method may include the step of analyzing the plasma sample for cathepsin S and determining the risk of cardiac injury in the human subject based on reduced cathepsin S concentration.

In some embodiments, the IgE-specific protein assay may be an enzyme-linked immunosorbent assay (ELISA).

In some embodiments, the IgE-specific protein assay may be a liquid chromatography mass spectrometry (LC-MS) assay

In some embodiments, the elevated IgE concentration may be determined as a measurement of IgE concentration in plasma, wherein the elevated IgE concentration in plasma may be selected from the group consisting of greater than 150 ng/mL, greater than 200 ng/mL, greater than 300 ng/mL, and greater than 400 ng/mL.

In some embodiments, the elevated IgE concentration may be determined as a measurement of IgE relative to immunoglobulin G1 (IgG1) in plasma (IgE/IgG1 ratio), wherein the IgE/IgG1 ratio may be selected from the group consisting of greater than 1.5×10⁻⁵, greater than 2×10⁻⁵, greater than 2.5×10⁻⁵, greater than 3×10⁻⁵, greater than 4×10⁻⁵, and greater than 5×10⁻⁵.

In another embodiment, the invention includes a method of preventing injury in a human subject being treated for cancer that includes the step of obtaining a plasma sample from the human subject. In some embodiments, the method includes the step of analyzing the plasma sample by an immunoglobulin E (IgE)-specific protein assay for IgE. In some embodiments, the method includes the step of determining whether the human subject may be at a high risk for cardiac injury from a chemotherapeutic regimen based on a reduced plasma IgE concentration. In some embodiments, the method may include the step of avoiding administration of a chemotherapeutic regimen, or selecting an alternate cancer therapy, to the human subject determined to have the high risk of cardiac injury.

In some embodiments, the cancer may be breast cancer.

In some embodiments, the method may include one or more of the steps of analyzing the plasma sample for beta-hydroxylase and determining the risk of cardiac injury in the human subject based on elevated beta-hydroxylase concentration.

In some embodiments, the method may include one or more of the steps of analyzing the plasma sample for cathepsin S and determining the risk of cardiac injury in the human subject based on elevated cathepsin S concentration.

In some embodiments, the IgE-specific protein assay may be an enzyme-linked immunosorbent assay (ELISA).

In some embodiments, the IgE-specific protein assay may be a liquid chromatography mass spectrometry (LC-MS) assay.

In some embodiments, the reduced IgE concentration may be determined as a measurement of IgE concentration in plasma, wherein the reduced IgE concentration in plasma may be selected from the group consisting of less than 100 ng/mL, less than 150 ng/mL, less than 200 ng/mL, and less than 250 ng/mL.

In some embodiments, the reduced IgE concentration may be determined as a measurement of IgE relative to immunoglobulin G1 (IgG1) in plasma (IgE/IgG1 ratio), wherein the IgE/IgG1 ratio may be selected from the group consisting of less than 1.5×10⁻⁵, less than 2×10⁻⁵, less than 2.5×10⁻⁵, less than 3×10⁻⁵, less than 4×10⁻⁵, and less than 5×10⁻⁵.

In another embodiment, the invention includes a kit for determining the risk of cardiac injury in a human subject receiving chemotherapy. The kit may include an assay for determining the concentration of immunoglobulin E (IgE) in plasma obtained from the human subject.

In some embodiments, the assay for determining the concentration of IgE may include an enzyme-linked immunosorbent assay (ELISA).

In some embodiments, the kit may include an assay for determining the concentration of immunoglobulin G1 (IgG1).

In some embodiments, the assay for determining the concentration of IgG1 may include an enzyme-linked immunosorbent assay (ELISA)

In some embodiments, the kit may include an assay for determining the concentration of beta-hydroxylase.

In some embodiments, the kit may include an assay for determining the concentration of cathepsin S.

### BRIEF DESCRIPTION OF THE DRAWINGS

The foregoing summary, as well as the following detailed description of the invention, will be better understood when read in conjunction with the appended drawings.

FIG. 1 illustrates the study cohort design and experimental approach. Blood draw and echocardiography protocol for patients who were treated with doxorubicin and trastuzumab therapy. A * denotes when transthoracic echocardiograms were performed. A [symbol] denotes when blood samples were collected.

FIG. 2 illustrates the strategy for proteomic discovery and validation of candidate biomarkers. Steps of Group A (highlighted in orange) were performed using longitudinal plasma samples (including baseline). Step B (highlighted in blue) was performed using only baseline plasma samples.

FIG. 3 illustrates longitudinal plots of ejection Fraction (EF) for cases and controls. Cases are shown in red, and controls in blue and green for controls 2A and 2B, respectively. Red arrows indicate the point of clinical diagnosis of cardiac dysfunction for each case. The selected subset of longitudinal plasma draws analyzed in this study are numbered (p1, p2 . . . , etc.) and indicated by solid markers. Note that blood draws typically precede echocardiogram measurements (open diamonds on the plots).

FIG. 4 illustrates top candidate predictive biomarkers. A heat map of relative protein levels, fold change differences and its significance between cases and controls is shown. Color-coded z-scores calculated for protein intensity for the top predictive markers are shown. Color scheme for individual plasma samples: red=high relative protein intensity; blue=low intensity; white=average intensity (no change); •=zero values that were replaced by imputed values for statistical analysis; #=not detected in this experiment. * Plasma samples analyzed. † p-value calculated from two-tailed Student's t-test. ‡Fold change between the average of all timepoints for each case and control pair. Red=fold change was >1.5-fold higher in cases; blue=fold change was >1.5-fold lower in cases.

FIGS. 5A to 5C illustrate longitudinal data from quantitative proteome analysis of three case and control pairs for the top three predictive markers, which have significant differences from the start of treatment and at all timepoints throughout the study. Inflection points indicate the positions of blood draws. FIG. 5A refers to Case 1/control 1. FIG. 5B refers to Case 2/Control 2A,2B. FIG. 5C refers to Case 3/Control 3. Red squares=point of clinical CTRCD diagnosis.

FIGS. 6A to 6G illustrate baseline levels of immunoglobulin subtypes using Luminex assays. Baseline measurements of IgE (FIG. 6A) and other Ig subtypes (FIGS. 6B to 6G) measured in the 3 case and 4 control samples from the proteomics discovery. Two-tailed Student's t-test p-values are reported.

FIGS. 7A and 7B illustrate ELISA validation for baseline IgE, IgG4, and IgG1 levels and ratios in the doxorubicin and trastuzumab cohort plasma. In FIG. 7A, standard sandwich ELISA results for baseline (prior to treatment) plasma for all 35 participants in the doxorubicin/trastuzumab cohort are shown. In FIG. 7B, ratios of the immunoglobulins assayed are shown. No CTRCD: participants treated with doxorubicin and trastuzumab who did not develop cancer therapy-related cardiac dysfunction. CTRCD: participants treated with doxorubicin and trastuzumab who were diagnosed with cancer therapeutics-related cardiac dysfunction. Wilcoxon Rank Sum test was used and corresponding p-values are reported. In each graph, the bars represent the median and interquartile range. The blue dotted line is the threshold observed which reaches the highest specificity and sensitivity for IgE or IgE/IgG1.

FIG. 8 illustrates ROC curves for IgE and IgE/IgG1 ratio. Receiver operator characteristic (ROC) curves for IgE and IgE/IgG1 for the prediction of CTRCD. The area under the ROC (AUC) curve is 0.73 for the log₂ IgE at baseline and 0.76 for the log₂ (IgE/IgG1) ratio.

FIG. 9 illustrates an experimental protocol for 3-D label-free quantitative biomarker discovery. “Top-20” depletion followed by SDS-PAGE and LC-MS/MS were used to compare longitudinal plasma samples from patients treated with doxorubicin and trastuzumab who were diagnosed with cancer therapeutics-related cardiac dysfunction (Cases) with their matched controls. MaxQuant label-free quantitation software was used to identify changes between the two groups.

FIGS. 10A to 10G illustrate longitudinal plots of immunoglobulin subtypes using Luminex assay. Longitudinal trends of IgE (FIG. 10A) and other Ig subtypes (FIGS. 10B to 10G) measured in 31 samples from the discovery case (red) and control (blue) pairs. Timepoints for each analyzed plasma draw are noted by symbols.

FIGS. 11 and 12A to 12F illustrate baseline levels of T-helper cell cytokines and chemokines using luminex assays. FIG. 11 illustrates baseline measurements of the 21 analytes measured in the 3 case and 4 control samples from the proteomics discovery. Two-tailed Student's t-test p value and fold change between averages are reported; heat map indicates highest values in red, lowest values in blue, and mid-range values in white for each analyte; zeroes indicate sample was below the detection limit of the assay; fold change >2.0 highlighted in green; *=because all case values were zero, average control value is reported for IL2. FIGS. 12A to 12F illustrate select analytes from FIG. 11 having p value <0.25 and fold change >2.0.

### BRIEF DESCRIPTION OF THE SEQUENCE LISTING

SEQ ID NO:1 is a heavy chain amino acid sequence for trastuzumab.

SEQ ID NO:2 is a light chain amino acid sequence for trastuzumab.

SEQ ID NO:3 is a variable heavy chain amino acid sequence for trastuzumab.

SEQ ID NO:4 is a variable light chain amino acid sequence for trastuzumab.

SEQ ID NO:5 is a variable heavy chain CDR1 amino acid sequence for trastuzumab.

SEQ ID NO:6 is a variable heavy chain CDR2 amino acid sequence for trastuzumab.

SEQ ID NO:7 is a variable heavy chain CDR3 amino acid sequence for trastuzumab.

SEQ ID NO:8 is a variable light chain CDR1 amino acid sequence for trastuzumab.

SEQ ID NO:9 is a variable light chain CDR2 amino acid sequence for trastuzumab.

SEQ ID NO:10 is a variable light chain CDR3 amino acid sequence for trastuzumab.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods of the invention.

The terms “active pharmaceutical ingredient” and “drug” refers to any compound that is biologically active, including individual drugs in the chemotherapeutic regimens described herein, such as doxorubicin, 5-fluorouracil, cyclophosphamide, paclitaxel, and trastuzumab.

The term “elevated concentration,” either as stated or in conjunction with the elevated concentration of a protein, antibody, or other relevant biomolecule (e.g., “elevated IgE concentration”), refers to a concentration of a protein, antibody, or other relevant biomolecule found in a human subject's bodily fluid (e.g., plasma) that is measurably greater than the concentration of the same protein, antibody, or other relevant biomolecule that is observed in a normal human subject's bodily fluid.

The term “reduced concentration,” either as stated or in conjunction with the reduced concentration of a protein, antibody, or other relevant biomolecule (e.g., “reduced IgE concentration”), refers to a concentration of a protein, antibody, or other relevant biomolecule found in a human subject's bodily fluid (e.g., plasma) that is measurably less than the concentration of the same protein, antibody, or other relevant biomolecule that is observed in a normal human subject's bodily fluid.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the human subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit in a human subject. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier,” “pharmaceutically acceptable excipient,” or “excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, Design of Prodrugs, Elsevier, Amsterdam, 1985). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, from 0% to 10%, from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or enantiomers can be prepared by asymmetric syntheses. See, for example, Jacques et al., Enantiomers, Racemates and Resolutions, Wiley Interscience, New York (1981); Eliel, Stereochemistry of Carbon Compounds, McGraw-Hill, New York (1962); and Eliel and Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

“Solvate” refers to a compound in physical association with one or more molecules of a pharmaceutically acceptable solvent.

Compounds used in the methods of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

The active pharmaceutical ingredients and/or drugs described herein also include antibodies. The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to, e.g., HER2 can be made using knowledge and skill in the art of injecting test subjects with HER2 antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion”), as used herein, refers to one or more fragments of an antibody that retain the ability to specifically bind to an antigen (e.g., HER2). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird et al., Science 1988, 242, 423-426; and Huston et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes. In mammals, there are five antibody isotypes: IgA, IgD, IgG, IgM and IgE. In humans, there are four subclasses of the IgG isotype: IgG1, IgG2, IgG3 and IgG4, and two subclasses of the IgA isotype: IgA1 and IgA2.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to a therapeutic moiety, such as a bacterial toxin, a cytotoxic drug or a radionuclide-containing toxin. Toxic moieties can be conjugated to antibodies of the invention using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 1986, 321, 522-525; Riechmann et al., Nature 1988, 332, 323-329; and Presta, Curr. Op. Struct. Biol. 1992, 2, 593-596.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki et al. Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino et al., Biochem. 1975, 14, 5516-5523.

The term “conservative amino acid substitutions” means amino acid sequence modifications which do not abrogate the binding of the antibody to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLO SUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-HER2 antibody is preferably replaced with another amino acid residue from the same class. Methods of identifying amino acid conservative substitutions which do not eliminate antigen binding are well-known in the art (see, e.g., Brummell et al., Biochemistry 1993, 32, 1180-1187; Kobayashi et al., Protein Eng. 1999, 12, 879-884 (1999); and Burks et al., Proc. Natl. Acad. Sci. USA 1997, 94, 412-417).

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

Certain embodiments of the invention comprise a variant of an antibody, e.g., an anti-HER2 antibody. As used herein, the term “variant” encompasses but is not limited to antibodies which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody.

The term “radioisotope-labeled complex” refers to both non-covalent and covalent attachment of a radioactive isotope, such as ⁹⁰Y, or ¹³¹I, to an antibody, including conjugates.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference anti-CD20 monoclonal antibody is rituximab, an anti-CD20 biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorised, approved for authorisation or subject of an application for authorisation under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorised by the EMA. However, in some instances, the biosimilar may be compared to a biological medicinal product which has been authorised outside the European Economic Area (a non-EEA authorised “comparator”) in certain studies. Such studies include for example certain clinical and in vivo non-clinical studies. As used herein, the term “biosimilar” also relates to a biological medicinal product which has been or may be compared to a non-EEA authorised comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorised or considered suitable for authorisation. In certain circumstances, the biosimilar exhibits different binding characteristics as compared to the reference medicinal product, wherein the different binding characteristics are considered by a Regulatory Authority such as the EMA not to be a barrier for authorisation as a similar biological product. The term “biosimilar” is also used synonymously by other national and regional regulatory agencies.

The term “binding molecule” as used herein includes molecules that contain at least one antigen binding site that specifically binds to HER2. By “specifically binds” it is meant that the binding molecules exhibit essentially background binding to molecules other than HER2. An isolated binding molecule that specifically binds HER2 may, however, have cross-reactivity to HER2 molecules from other species.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, ALL, CLL, SLL, acute myelogenous leukemia (AML), chronic myelogenous leukemia (CIVIL), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### Methods of Treating Cancers and Other Diseases

The compositions and methods described herein can be used in a method for treating diseases. In an embodiment, they are for use in treating hyperproliferative disorders. They may also be used in treating other disorders as described herein and in the following paragraphs.

In some embodiments, the methods described herein may include treating a hyperproliferative disorder (e.g., cancer or breast cancer) in a human subject, wherein the human subject exhibits an elevated concentration of immunoglobulin (Ig) in plasma obtained from the human subject.

In some embodiments, the methods described herein may include treating a hyperproliferative disorder (e.g., cancer or breast cancer) in a human subject, wherein the human subject exhibits an elevated concentration of immunoglobulin E (IgE) in plasma obtained from the human subject. In some embodiments, the elevated concentration of IgE may be determined as a measurement of IgE concentration in plasma, wherein the IgE concentration in plasma is greater than about 150 ng/mL, or greater than about 200 ng/mL, or greater than about 300 ng/mL, or greater than about 400 ng/mL.

In some embodiments, the methods described herein may include treating a hyperproliferative disorder (e.g., cancer) in a human subject, wherein the human subject exhibits an elevated concentration of immunoglobulin G1 (IgG1) in plasma obtained from the human subject.

In some embodiments, the methods described herein may include treating a hyperproliferative disorder (e.g., cancer) in a human subject, wherein the human subject exhibits an elevated concentration of immunoglobulin E (IgE) relative to immunoglobulin G1 (IgG1) in plasma (IgE/IgG1 ratio), wherein the IgE/IgG1 ratio is greater than about 1.5×10⁻⁵, or greater than about 2×10⁻⁵, or greater than about 2.5×10⁻⁵, or greater than about 3×10⁻⁵, or greater than about 4×10⁻⁵, or greater than about 5×10⁻⁵.

In some embodiments of the methods described herein, the hyperproliferative disorder (e.g., cancer) may be breast cancer.

In some embodiments of the methods described herein, the human subject may further exhibit a reduced concentration of beta-hydroxylase in plasma obtained from the human subject. In certain embodiments, the methods may include determining the risk of cardiac injury in the human subject based on reduced beta-hydroxylase concentration.

In some embodiments of the methods described herein, the human subject may further exhibit a reduced concentration of cathepsin S in plasma obtained from the human subject. In certain embodiments, the methods may include determining the risk of cardiac injury in the human subject based on reduced cathepsin S concentration.

In some embodiments, the methods described herein may include determining whether a human subject is at a low risk for cardiac injury from a chemotherapeutic regiment based on an elevated IgE concentration. In some embodiments, the methods described herein may include administering a chemotherapeutic regimen to a human subject determined to have the low risk of cardiac injury.

In some embodiments, the invention includes methods of preventing injury, such as cardiac injury, in a human subject being treated for cancer. In some embodiments, the methods may include determining whether the human subject may be at a high risk for cardiac injury from a chemotherapeutic regiment based on a reduced IgE concentration.

In some embodiments, the methods may include the determination of reduced IgE concentration as a measurement of IgE concentration in plasma, where the reduced IgE concentration in plasma may be less than about 100 ng/mL, or less than about 150 ng/mL, or less than about 200 ng/mL, or less than about 250 ng/mL.

In some embodiments, the methods may include the determination of reduced IgE concentration as a measurement of IgE relative to IgG1 in plasma (IgE/IgG1 ratio), where the reduced IgE/IgG1 ratio may be less than about 1.5×10⁻⁵, or less than about 2×10⁻⁵, or less than about 2.5×10⁻⁵, or less than about 3×10⁻⁵, or less than about 4×10⁻⁵, or less than about 5×10⁻⁵.

In some embodiments, the methods may include preventing administration of a chemotherapeutic regimen to the human subject determined to have a high risk of cardiac injury.

In some embodiments of the methods or kits described herein, the cancer may be breast cancer. In some embodiments of the methods or kits described herein, the cancer may be HER2-positive (HER2⁺) breast cancer. In some embodiments of the methods or kits described herein, the cancer may be HER2-negative (HER2⁻) breast cancer. HER2 status may be confirmed by standard diagnostic tests known in the art.

In some embodiments of the methods described herein, the cardiac injury includes cancer therapeutics-related cardiac dysfunction (CTRCD).

The methods described herein may include one or more of the steps of obtaining a plasma sample from a human subject and analyzing the plasma sample by an immunoglobulin (Ig)-specific protein assay. In some embodiments, the immunoglobulin (Ig)-specific protein assay includes an immunoglobulin E (IgE)-protein specific assay. In some embodiments, the immunoglobulin (Ig)-specific protein assay includes an immunoglobulin G1 (IgG1)-protein specific assay.

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany et al., Endocrinology 2012, 153, 1585-92; and Fong et al., J. Ovarian Res. 2009, 2, 12. Models for determining efficacy of treatments for pancreatic cancer are described in Herreros-Villanueva et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for melanoma are described, e.g., in Damsky et al., Pigment Cell & Melanoma Res. 2010, 23, 853-859. Models for determining efficacy of treatments for lung cancer are described, e.g., in Meuwissen et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy in B cell lymphomas, such as diffuse large B cell lymphoma (DLBCL), include the PiBCL1 murine model with BALB/c (haplotype H-2d) mice. Illidge et al., Cancer Biother. & Radiopharm. 2000, 15, 571-80. Efficacy of treatments for Non-Hodgkin's lymphoma may be assessed using the 38C13 murine model with C3H/HeN (haplotype 2-Hk) mice or alternatively the 38C13 Her2/neu model. Timmerman et al., Blood 2001, 97, 1370-77; Penichet et al., Cancer Immunolog. Immunother. 2000, 49, 649-662. Efficacy of treatments for chronic lymphocytic leukemia (CLL) may be assessed using the BCL1 model using BALB/c (haplotype H-2d) mice. Dutt et al., Blood 2011, 117, 3230-29.

### Chemotherapeutic Regimens

In an embodiment, the invention includes a method of treating a cancer in a human subject, wherein the human subject exhibits an elevated concentration of immunoglobulin E (IgE) in plasma obtained from the human subject, comprising the step of administering a therapeutically effective amount of a chemotherapeutic regimen to the human subject in need thereof.

The chemotherapeutic regimens described herein may include any of the following non-limiting active pharmaceutical ingredients.

In an embodiment, the chemotherapeutic regimen includes trastuzumab, or a fragment, derivative, conjugate, variant, radioisotope-labeled complex, or biosimilar thereof. Trastuzumab is a recombinant humanized version of the murine anti-HER2 antibody 4D5, and is also known as huMAb4D5-8, rhuMAb HER2, or HERCEPTIN. The preparation and properties of trastuzumab are known in the art and are described, e.g., in U.S. Pat. No. 5,821,337, the disclosure of which is incorporated by reference herein. Trastuzumab is commercially available from multiple suppliers including Roche (Genentech). Trastuzumab active in preclinical models as well as clinically active in patients with HER2-overexpressing metastatic breast cancers that have received extensive prior anti-cancer therapy. Baselga et al., J. Clin. Oncol. 1996, 14, 737-744; Molina et al., Cancer Res. 2001, 61, 4744-4749. Trastuzumab received marketing approval from the Food and Drug Administration in 1998 for the treatment of patients with metastatic breast cancer whose tumors overexpress the HER2 protein. The amino acid sequence for the heavy chain of trastuzumab is set forth in SEQ ID NO:1. The amino acid sequence for the light chain of trastuzumab is set forth in SEQ ID NO:2.

In an embodiment, the chemotherapeutic regimen includes an anti-HER2 monoclonal antibody, such as trastuzumab, or a fragment, derivative, conjugate, variant, radioisotope-labeled complex, or biosimilar thereof. In some embodiments, the anti-HER2 monoclonal antibody comprises a heavy chain comprising SEQ ID NO:1 and a light chain comprising SEQ ID NO:2. In an embodiment, the anti-HER2 monoclonal antibody has a heavy chain sequence identity of greater than 90% to SEQ ID NO:1. In an embodiment, the anti-HER2 monoclonal antibody has a light chain sequence identity of greater than 90% to SEQ ID NO:2. In an embodiment, the anti-HER2 monoclonal antibody has a heavy chain sequence identity of greater than 95% to SEQ ID NO:1. In an embodiment, the anti-HER2 monoclonal antibody has a light chain sequence identity of greater than 95% to SEQ ID NO:2. In an embodiment, the anti-HER2 monoclonal antibody has a heavy chain sequence identity of greater than 98% to SEQ ID NO:1. In an embodiment, the anti-HER2 monoclonal antibody has a light chain sequence identity of greater than 98% to SEQ ID NO:2. In an embodiment, the anti-HER2 monoclonal antibody has a heavy chain sequence identity of greater than 99% to SEQ ID NO:1. In an embodiment, the anti-HER2 monoclonal antibody has a light chain sequence identity of greater than 99% to SEQ ID NO:2.

In some embodiments, the anti-HER2 monoclonal antibody comprises a heavy chain comprising the variable region in SEQ ID NO:3 and a light chain comprising the variable region in SEQ ID NO:4. In an embodiment, the anti-HER2 antibody comprises a heavy chain variable region comprising SEQ ID NO:3. In an embodiment, the anti-HER2 antibody comprises a light chain variable region comprising SEQ ID NO:4. In an embodiment, the anti-HER2 monoclonal antibody has a variable heavy chain sequence identity of greater than 90% to SEQ ID NO:3. In an embodiment, the anti-HER2 monoclonal antibody has a variable light chain sequence identity of greater than 90% to SEQ ID NO:4. In an embodiment, the anti-HER2 monoclonal antibody has a variable heavy chain sequence identity of greater than 95% to SEQ ID NO:3. In an embodiment, the anti-HER2 monoclonal antibody has a variable light chain sequence identity of greater than 95% to SEQ ID NO:4. In an embodiment, the anti-HER2 monoclonal antibody has a variable heavy chain sequence identity of greater than 98% to SEQ ID NO:3. In an embodiment, the anti-HER2 monoclonal antibody has a variable light chain sequence identity of greater than 98% to SEQ ID NO:4. In an embodiment, the anti-HER2 monoclonal antibody has a variable heavy chain sequence identity of greater than 99% to SEQ ID NO:3. In an embodiment, the anti-HER2 monoclonal antibody has a variable light chain sequence identity of greater than 99% to SEQ ID NO:4.

In an embodiment, the anti-HER2 antibody comprises a heavy chain CDR selected from the group consisting of SEQ ID NO:5, SEQ ID NO:6 and SEQ ID NO:7, or conservative amino acid substitutions thereof. In an embodiment, the anti-HER2 antibody comprises a heavy chain CDR1 comprising the amino acid sequence of SEQ ID NO:5, or conservative amino acid substitutions thereof. In an embodiment, the anti-HER2 antibody comprises a heavy chain CDR2 comprising the amino acid sequence of SEQ ID NO:6, or conservative amino acid substitutions thereof. In an embodiment, the anti-HER2 antibody comprises a heavy chain CDR3 comprising the amino acid sequence of SEQ ID NO:7, or conservative amino acid substitutions thereof. In an embodiment, the anti-HER2 antibody comprises one or more heavy chain CDRs selected from the group consisting of SEQ ID NO:5, SEQ ID NO:6, and SEQ ID NO:7, or conservative amino acid substitutions thereof. In an embodiment, the anti-HER2 antibody comprises a heavy chain CDR1 comprising the amino acid sequence of SEQ ID NO:5, a heavy chain CDR2 comprising the amino acid sequence of SEQ ID NO:6, and a heavy chain CDR3 comprising the amino acid sequence of SEQ ID NO:7, or conservative amino acid substitutions thereof.

In an embodiment, the anti-HER2 antibody comprises a light chain CDR selected from the group consisting of SEQ ID NO:8, SEQ ID NO:9 and SEQ ID NO:10, or conservative amino acid substitutions thereof. In an embodiment, the anti-HER2 antibody comprises a light chain CDR1 comprising the amino acid sequence of SEQ ID NO:8, or conservative amino acid substitutions thereof. In an embodiment, the anti-HER2 antibody comprises a light chain CDR2 comprising the amino acid sequence of SEQ ID NO:9, or conservative amino acid substitutions thereof. In an embodiment, the anti-HER2 antibody comprises a light chain CDR3 comprising the amino acid sequence of SEQ ID NO:10, or conservative amino acid substitutions thereof. In an embodiment, the anti-HER2 antibody comprises one or more light chain CDRs selected from the group consisting of SEQ ID NO:8, SEQ ID NO:9, and SEQ ID NO:10, or conservative amino acid substitutions thereof. In an embodiment, the anti-HER2 antibody comprises a light chain CDR1 comprising the amino acid sequence of SEQ ID NO:8, a light chain CDR2 comprising the amino acid sequence of SEQ ID NO:9, and a light chain CDR3 comprising the amino acid sequence of SEQ ID NO:10, or conservative amino acid substitutions thereof.

In an embodiment, the anti-HER2 antibody is a biosimilar monoclonal antibody approved by drug regulatory authorities with reference to trastuzumab. In an embodiment, the biosimilar comprises an anti-HER2 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is trastuzumab. In some embodiments, the one or more post-translational modifications are selected from one or more of: glycosylation, oxidation, deamidation, and truncation. In some embodiments, the biosimilar is a trastuzumab antibody authorized or submitted for authorization, wherein the anti-HER2 antibody is provided in a formulation which differs from the formulations of a reference medicinal product or reference biological product, wherein the reference medicinal product or reference biological product is trastuzumab. The anti-HER2 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is trastuzumab. In some embodiments, the biosimilar comprises one or more excipients selected from tris-hydrochloride, sodium chloride, mannitol, pentetic acid, polysorbate 80, sodium hydroxide, and hydrochloric acid.

The amino acid sequences of trastuzumab and related antibodies, including biosimilar antibodies, referenced in the foregoing are summarized in Table 1.

TABLE 1
Trastuzumab and related anti-HER2 antibody amino acid sequences.

| Sequence | | |
|---|---|---|
| Identifier and | | |
| Description | Sequence (One-Letter Amino Acid Symbols) | |
| SEQ ID NO: 1 | EVQLVESGGG LVQPGGSLRL SCAASGFNIK DTYIHWVRQA PGKGLEWVAR IYPTNGYTRY | 60 |
| trastuzumab | ADSVKGRFTI SADTSKNTAY LQMNSLRAED TAVYYCSRWG GDGFYAMDYW GQGTLVTVSS | 120 |
| heavy chain | ASTKGPSVFP LAPSSKSTSG GTAALGCLVK DYFPEPVTVS WNSGALTSGV HTFPAVLQSS | 180 |
| | GLYSLSSVVT VPSSSLGTQT YICNVNHKPS NTKVDKKVEP PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRD | 360 |
| | ELTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 2 | DIQMTQSPSS LSASVGDRVT ITCRASQDVN TAVAWYQQKP GKAPKLLIYS ASFLYSGVPS | 60 |
| trastuzumab | RFSGSRSGTD FTLTISSLQP EDFATYYCQQ HYTTPPTFGQ GTKVEIKRTV AAPSVFIFPP | 120 |
| light chain | SDEQLKSGTA SVVCLLNNFY PREAKVQWKV DNALQSGNSQ ESVTEQDSED STYSLSSTLT | 180 |
| | LSKADYEKHK VYACEVTHQG LSSPVTKSFN RGEC | 214 |
| SEQ ID NO: 3 | EVQLVESGGG LVQPGGSLRL SCAASGFNIK DTYIHWVRQA PGKGLEWVAR IYPTNGYTRY | 60 |
| trastuzumab | ADSVKGRFTI SADTSKNTAY LQMNSLRAED TAVYYCSRWG GDGFYAMDYW GQGTLVT | 117 |
| variable heavy | | |
| chain | | |
| SEQ ID NO: 4 | DIQMTQSPSS LSASVGDRVT ITCRASQDVN TAVAWYQQKP GKAPKLLIYS ASFLYSGVPS | 60 |
| trastuzumab | RFSGSRSGTD FTLTISSLQP EDFATYYCQQ HYTTPPTFGQ GTK | 103 |
| variable light | | |
| chain | | |
| SEQ ID NO: 5 | GFNIKDTYIH | 10 |
| trastuzumab | | |
| variable heavy | | |
| chain CDR1 | | |
| SEQ ID NO: 6 | RIYPTNGYTR YADSVKG | 17 |
| trastuzumab | | |
| variable heavy | | |
| chain CDR2 | | |
| SEQ ID NO: 7 | WGGDGFYAMD Y | 11 |
| trastuzumab | | |
| variable heavy | | |
| chain CDR3 | | |
| SEQ ID NO: 8 | RASQDVNTAV A | 11 |
| trastuzumab | | |
| variable light | | |
| chain CDR1 | | |
| SEQ ID NO: 9 | SASFLYS | 7 |
| trastuzumab | | |
| variable light | | |
| chain CDR2 | | |
| SEQ ID NO: 10 | QQHYTTPPT | 9 |
| trastuzumab | | |
| variable light | | |
| chain CDR3 | | |

Other anti-HER2 antibodies suitable for use in the chemotherapeutic regimens described herein have been described in Tagliabue et al., Mt. Cancer 1991, 47, 933-937; McKenzie et al., Oncogene 1989, 4, 543-548; Maier et al., Cancer Res. 1991, 51, 5361-5369; Bacus et al., Molecular Carcinogenesis 1990, 3, 350-362; Stancovski et al., Proc. Natl. Acad. Set. USA 1991, 88, 8691-8695; Bacus et al., Cancer Res. 1992, 52, 2580-2589; Xu et al., Int. Cancer 1993, 53, 401-408; Kasprzyk et al., Cancer Res. 1992, 52, 2771-2776 (1992); Hancock et al., Cancer Res. 1991, 51, 4575-4580; Shawver et al., Cancer Res. 1994, 54, 1367-1373; Arteaga et al., Cancer Res. 1994, 54, 3758-3765; Harwerth et al., J. Biol. Chern. 1992, 267, 15160-15167; and Klapper et al., Oncogene 1997, 14, 2099-2109. Additional anti-HER2 antibodies suitable for use in the chemotherapeutic regimens described herein are described in U.S. Pat. No. 5,783,186, the disclosure of which is incorporated by reference herein.

In an embodiment, the chemotherapeutic regimen includes doxorubicin, which has the chemical name (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Formula (1)):

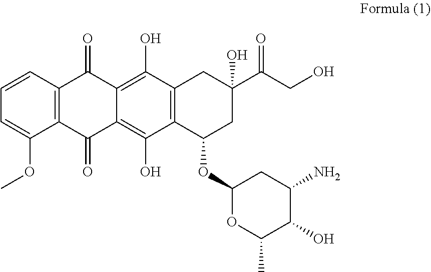 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

or a pharmaceutically acceptable salt, solvate, hydrate, cocrystal, tautomer, diastereomer, or prodrug thereof. In an embodiment, the chemotherapeutic regimen includes doxorubicin hydrochloride. Doxorubicin is commercially available from multiple suppliers, and is sold under trade names such as ADRIAMYCIN. The preparation and properties of doxorubicin are known in the art and is also described in U.S. Pat. Nos. 3,590,028 and 4,012,448, the disclosures of which are incorporated herein by reference. In an embodiment, the chemotherapeutic regimen includes liposomal doxorubicin, which is commercially available under trade names such as CAELYX, MYOCET, or DOXIL, and is described in, e.g., Waterhouse et al., Drug Saf 2001, 24, 903-20.

In an embodiment, the chemotherapeutic regimen includes cyclophosphamide, which is also known as cytophosphane, and which has the chemical name (RS)—N,N-bis(2-chloroethyl)-1,3,2-oxazaphosphinan-2-amine 2-oxide (Formula (2)):

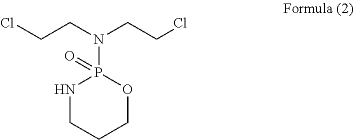 [SMILES: O=P1(N(CCCl)CCCl)NCCCO1]

or a pharmaceutically acceptable salt, solvate, hydrate, cocrystal, tautomer, enantiomer, or prodrug thereof. Cyclophosphamide is commercially available from multiple suppliers under various trade names, including CYCLOBLASTIN, CYCLOSTIN, CYTOXAN, ENDOXAN, PROCYTOX, and SENDOXAN. The synthesis and properties of cyclophosphamide are known in the art, and are described, e.g., in Arnold et al., Naturewiss. 1957, 45, 64 and Arnold et al., Angew. Chem. 1958, 70, 539 and U.S. Pat. No. 3,018,302, the disclosure of which is incorporated herein by reference.

In an embodiment, the chemotherapeutic regimen includes 5-fluorouracil, which is also known as ADRUCIL, and which has the chemical name (RS)—N,N-bis(2-chloroethyl)-1,3,2-oxazaphosphinan-2-amine 2-oxide (Formula (3)):

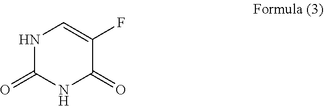

or a pharmaceutically acceptable salt, solvate, hydrate, cocrystal, tautomer, or prodrug thereof. 5-Fluorouracil is commercially available from multiple suppliers under various trade names, including ADRUCIL, ARUMEL, EFUDEX, EFUDIX, FLURIL, FLURACIL, FLUROPLEX, FLUROBLASTIN, and TIMAZIN. The synthesis and properties of 5-fluorouracil are known in the art and are described, e.g., in U.S. Pat. Nos. 2,802,005 and 2,885,396, the disclosures of which are incorporated by reference herein.

In an embodiment, the chemotherapeutic regimen includes paclitaxel, which is also known as TAXOL, and which has the chemical name (2α, 4α, 5β, 7β, 10β, 13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate (Formula (4)):

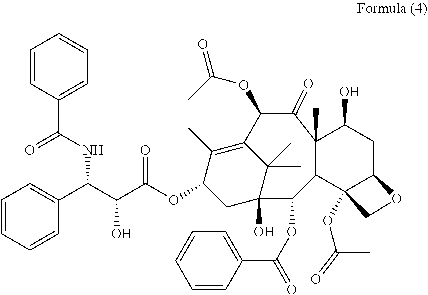 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

or a pharmaceutically acceptable salt, solvate, hydrate, cocrystal, tautomer, diastereomer, or prodrug thereof. Paclitaxel is commercially available from multiple suppliers under various trade names including TAXOL, ANZATAX, and PAXENE. The synthesis and properties of paclitaxel are known in the art and are described in Holton et al., J. Am. Chem. Soc. 1994, 116, 1597 and Nicolou et al., Nature 1994, 367, 630. In an embodiment, the chemotherapeutic regimen includes albumin-bound paclitaxel, which is commercially available under trade names such as ABRAXANE.

In an embodiment, the chemotherapeutic regimen includes doxorubicin monotherapy. Doxorubicin monotherapy, including low-dose doxorubcin monotherapy (8-12 mg/m² doxombicin/week), is known in the art and is described, e.g., in Scheithauer et al., Breast Cancer Res. & Treatment, 1985, 6, 89-93.

In an embodiment, the chemotherapeutic regimen includes trastuzumab monotherapy. Trastuzumab monotherapy is known in the art and is described in Nishimura et al., Breast Cancer 2008, 15, 57-64.

In an embodiment, the chemotherapeutic regimen includes doxorubicin and trastuzumab combination therapy. Doxorubicin and trastuzumab combination therapy is known in the art and has been described, e.g., in Rayson et al., Ann. Oncol. 2008, 19, 1530-1539.

In an embodiment, the chemotherapeutic regimen includes doxorubicin, cyclophosphamide, and 5-fluorouracil combination therapy. In an embodiment, the chemotherapeutic regimen includes treatment with doxorubicin at a dose of 50 mg/m² intravenously on day 1, 5-fluorouracil at a dose of 500 mg/m² intravenously on days 1 and 8 (1000 mg/m² each cycle), and cyclophosphamide 500 mg/m² intravenously on days 1 and 8, where the cycle is repeated every 21 days for three cycles. Doxorubicin, cyclophosphamide, and 5-fluorouracil combination therapy is known in the art and have been described, e.g., in Maloisel et al., Cancer. 1990, 65, 851-855.

In an embodiment, the chemotherapeutic regimen includes doxorubicin, cyclophosphamide, paclitaxel, and trastuzumab combination therapy. In an embodiment, the chemotherapeutic regimen includes initial treatment with doxorubicin and cyclophosphamide followed by treatment with paclitaxel and trastuzumab (ACTH chemotherapy). In an embodiment, the chemotherapeutic regimen includes doxorubicin (60 mg/m²) i.v. and cyclophosphamide (600 mg/m2) repeated every 21 days for 4 cycles, followed by paclitaxel 80 mg/m² via i.v. infusion weekly for 12 weeks, with trastuzumab 4 mg/kg i.v. with first dose of paclitaxel and trastuzumab 2 mg/kg i.v. weekly for the remainder of the 12 week period. In an embodiment, the chemotherapeutic regimen includes trastuzumab 6 mg/kg i.v. every 21 days may be used following the completion of paclitaxel treatment for a total of 1 year of trastuzumab treatment. ACTH chemotherapy protocols are known in the art and have been described, e.g., in Crozier et al., World J. Clin. Oncol. 2014, 5, 529-538.

In an embodiment, the chemotherapeutic regimen includes docetaxel, carboplatin, and trastuzumab combination therapy (i.e., TCH therapy). In an embodiment, the chemotherapeutic regimen includes a treatment (e.g., a 3 week cycle) of trastuzumab plus carboplatin at area under the serum concentration-time curve 6 and docetaxel at 75 mg/m², with trastuzumab given at 4 mg/kg as a loading dose followed by a 2 mg/kg dose once per week during the treatment every 2 weeks. TCH chemotherapy protocols are known in the art and have been described, e.g., in Valero et al., JCO. 2010, 29, 149-156.

In an embodiment, the chemotherapeutic regimen includes 5-fluorouracil monotherapy. In an embodiment, the chemotherapeutic regimen includes a treatment with 5-fluorouracil at 12 mg/kg/day for 5 days followed by 6 mg/kg on alternate days to slight toxicity or until 11 half doses are administered. 5-fluorouracil chemotherapy protocols are known in the art and have been described, e.g., in Ansfield, Cancer. 1977, 39, 34-40.

### Pharmaceutical Compositions and Routes of Administration

In an embodiment, an active pharmaceutical ingredient or combination of active pharmaceutical ingredients, such as any of the foregoing chemotherapeutic regimens, is provided as a pharmaceutically acceptable composition.

In some embodiments, the concentration of each of the active pharmaceutical ingredients provided in the pharmaceutical compositions of the invention, such as any of the foregoing chemotherapeutic regimens, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the invention, such as any of the foregoing chemotherapeutic regimens, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the invention, such as any of the foregoing chemotherapeutic regimens, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the invention, such as any of the foregoing chemotherapeutic regimens, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the invention, such as any of the foregoing chemotherapeutic regimens, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the invention, such as any of the foregoing chemotherapeutic regimens, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the foregoing chemotherapeutic regimens may also be used if appropriate.

In an embodiment, the molar ratio of two active pharmaceutical ingredients in the pharmaceutical compositions is in the range from 10:1 to 1:10, from 2.5:1 to 1:2.5, and about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

Described below are non-limiting pharmaceutical compositions and methods for preparing the same.

In some embodiments, the invention provides a pharmaceutical composition for oral administration containing the active pharmaceutical ingredient or combination of active pharmaceutical ingredients, such as the chemotherapeutic regimens described herein, and a pharmaceutical excipient suitable for oral administration.

In some embodiments, the invention provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the invention provides a pharmaceutical composition for injection containing an active pharmaceutical ingredient or combination of active pharmaceutical ingredients, such as an active pharmaceutical ingredient in the chemotherapeutic regimens described herein, and a pharmaceutical excipient suitable for injection.

The forms in which the compositions of the invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions of the chemotherapeutic regimens described herein may also be prepared from compositions described herein and one or more pharmaceutically acceptable excipients suitable for topical, inhalation, sublingual, buccal, rectal, intraosseous, intraocular, intranasal, epidural, or intraspinal administration. Preparations for such pharmaceutical compositions are well-known in the art. See, e.g., Anderson et al., eds., Handbook of Clinical Drug Data, Tenth Edition, McGraw-Hill, 2002; and Pratt and Taylor, eds., Principles of Drug Action, Third Edition, Churchill Livingston, 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), via local delivery by catheter or stent or through inhalation. The active pharmaceutical ingredient or combination of active pharmaceutical ingredients can also be administered intrathecally.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. In some embodiments, the kits may be provided for determining risk of cardiac injury (e.g., CTRCD) in a human subject receiving chemotherapy. In some embodiments, the kits may include an assay for determining the concentration of one or more of IgE, IgG1, beta-hydroxylase, and/or cathepsin S, in plasma obtained from a human subject. Such assays may include, without limitation, enzyme-linked immunosorbent assays (ELISA).

In some embodiments, the kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the invention provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are for use in the treatment of the diseases and conditions described herein. In an embodiment, the kits are for use in the treatment of cancer. In some embodiments, the kits are for use in treating solid tumor cancers.

In an embodiment, the kits of the invention are for use in the treatment of cancer or for the detection of cancer biomarkers. In an embodiment, the kits of the invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In an embodiment, the kits of the invention are for use in the treatment of breast cancer. In some embodiments, the cancer may be HER2-positive (HER2⁺) breast cancer. In some embodiments, the cancer may be HER2-negative (HER2⁻) breast cancer. HER2 status may be confirmed by standard diagnostic tests known in the art.

### Dosages and Dosing Regimens

The amounts of the pharmaceutical compositions administered using the methods herein, such as the dosages and/or amounts of chemotherapeutic regimens, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients in the methods of the invention may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an active pharmaceutical ingredient disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Study Population

Cases and controls were selected from the Cardiotoxicity of Cancer Therapy (CCT) study, an ongoing, National Heart Lung and Blood Institute (NHLBI)-funded prospective longitudinal cohort study of women with breast cancer recruited from the Rena Rowan Breast Cancer Center of the Abramson Cancer Center at the University of Pennsylvania (Philadelphia, Pa.). The primary inclusion criteria were women at least 18 years of age diagnosed with breast cancer and prescribed doxorubicin and/or trastuzumab therapy. The only exclusion criterion was pregnancy. Cases and controls all received doxorubicin (240 mg/m²) and cyclophosphamide followed by paclitaxel and trastuzumab, the latter as per standard prescribing algorithms.

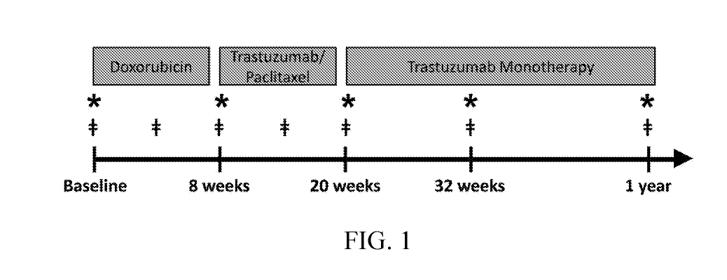

At baseline, prior to initiation of chemotherapy, and at each follow-up visit, each participant provided detailed clinical data via standardized questionnaires. Clinical data were verified via review of medical records. Blood samples were obtained at baseline, during doxorubicin, after doxorubicin completion, and every 6 weeks during trastuzumab. Transthoracic echocardiograms were performed at standardized intervals, and participants underwent an echocardiogram at baseline, after doxorubicin completion, and every 3 months during trastuzumab therapy (FIG. 1). This study was approved by the University of Pennsylvania Institutional Review Board, and all participants provided written informed consent.

### Example 2—Transthoracic Echocardiography

Transthoracic echocardiograms were acquired by a dedicated sonographer team at an Intersocietal Accreditation Commission laboratory according to a specific protocol at baseline and standardized time intervals. Two-dimensional images were acquired using Vivid 7 or E9 machines (GE Healthcare, Milwaukee, Wis.).

Echocardiograms were quantitated at the University of Pennsylvania Center for Quantitative Echocardiography (Philadelphia, Pa.). Quantitation was performed using the Tomtec Cardiac Performance Analysis (Unterschleissheim, Germany). Apical 4-chamber LV end-diastolic volume (EDV) and end-systolic volume (ESV) were calculated using the Simpson's method of discs as recommended by the American Society of Echocardiography (ASE), and used to derive left ventricular ejection fraction. Lang et al., J. Am. Soc. Echocardiogr. 2015, 28, 1-39 e14.

### Example 3—Identification of Cancer Therapy Related Cardiac Dysfunction Cases and Controls

Of the participants who experienced CTRCD, as defined as cardiac dysfunction with a reduction in EF by ≥10% from baseline to an absolute value of <50% at any subsequent visit, three were selected for proteomics analyses. Cases also had to experience symptoms of heart failure as adjudicated by a cardiologist, and all were started on cardiac medications.

Controls were selected based upon the lack of significant EF change during the entire duration of follow-up (<10% absolute change in EF and EF>50%) were matched to the cases based upon specific selection criteria. These criteria for matching included: age (+/−10 years), hormonal status, cancer stage, and race. In one instance, a patient meeting all of the criteria was not available. For this reason, two controls were selected for Case 2 (Table 2).

TABLE 2
Clinical Characteristics of Case/Control Pairs used in Discovery Proteomics.

| | | | | | Breast | | Nadir | CTRCD | |
|---|---|---|---|---|---|---|---|---|---|
| | Age | | Nodal | Hormone | Cancer | Baseline | EF | Timing | Cardiac |
| Patient | (yrs) | Race | Status | Status | Side | EF (%) | (%) | (days) | Medication |
| Case 1 | 48 | Cauc | N1 | ER+ | Right | 57 | 37 | 238 | Enalapril, |
| | | | | | | | | | Carvedilol |
| Ctrl 1 | 40 | Cauc | N1 | ER+ | Right | 63 | 55 | | None |
| Case 2 | 46 | Cauc | N3C | ER− | Right | 53 | 42 | 226 | Enalapril, |
| | | | | | | | | | Carvedilol |
| Ctrl 2A | 43 | Asian | N2 | ER− | Left | 58 | 53 | | None |
| Ctrl 2B | 53 | Cauc | N2 | ER+ | Left | 50 | 45 | | None |
| Case 3 | 43 | Asian | N0 | ER− | Right | 53 | 38 | 167 | Lisinopril, |
| | | | | | | | | | Carvedilol |
| Ctrl 3 | 36 | Cauc | N0 | ER− | Left | 61 | 52 | | None |

### Example 4—Plasma Sample Collection, Timepoints, and Processing

For all participants, blood was collected from venipuncture in the presence of EDTA, and this plasma was processed at 3353 RPM for 20 minutes, aliquoted and stored at −80° C. Longitudinal plasma samples for each case and control were selected for the proteomics study to evaluate changes over time in the proteome between cases and controls. In total, 31 samples from three cases and four controls in the cohort of participants receiving doxorubicin followed by trastuzumab were selected for discovery analyses. Samples used in the discovery analysis were derived from the following timepoints: prior to any chemotherapy, during chemotherapy, at the time of CTRCD diagnosis, and after the CTRCD diagnosis (FIG. 1). Timepoints for the matched controls were selected to match the case specimens. Samples used in subsequent validation analysis were derived from baseline only.

### Example 5—Proteomics Discovery and Data Analysis

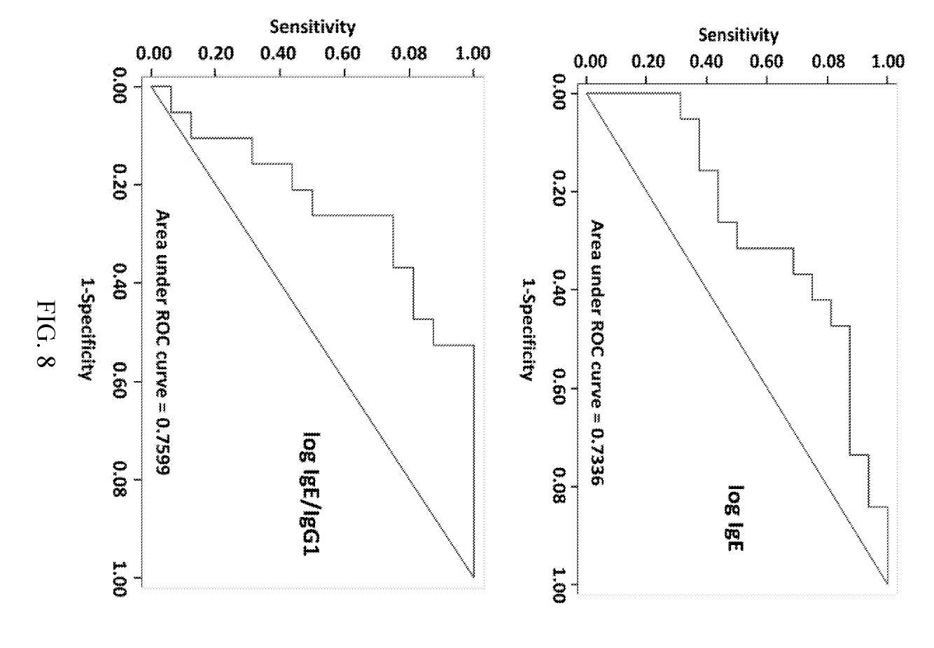

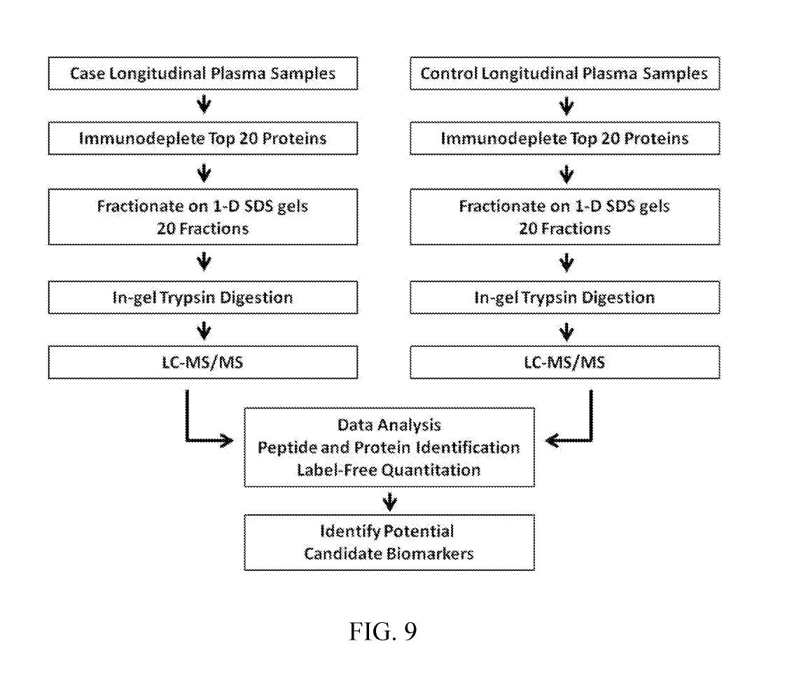

The 31 longitudinal case and control plasma samples used for discovery were processed using a 3-dimensional plasma proteome analysis strategy previously developed by the Speicher laboratory. Beer et al., J. Proteome Res. 2011, 10, 1126-1138. See FIG. 9. Briefly, 80 μl aliquots of plasma samples were depleted of 20 abundant plasma proteins using a ProteoPrep20 Immunodepletion Column (Sigma-Aldrich, St. Louis, Mo.) and run for a short distance (2 cm) on 1-D SDS gels. Plasma samples were filtered through a 0.22 μm microcentrifuge filter and injected onto the immunodepletion column. The flow-through fractions containing unbound proteins were collected, pooled, and precipitated overnight with 200 proof ethanol that was prechilled to −20° C. Ethanol supernatants were carefully removed and protein pellets were frozen and stored at −20° C. Frozen protein pellets were thawed briefly, resuspended in SDS-PAGE sample buffer, reduced and alkylated, and run for a short distance (2 cm) on 1-D SDS gels. Three lanes representing the immunodepleted fraction from 10 μl of original plasma were run for each sample, and each lane was sliced into twenty 1-mm slices. Corresponding slices for the triplicate lanes of each sample that represented a total of 30 μl original plasma were combined and digested with trypsin.

Each set of case/control samples was analyzed in a separate label-free LC-MS/MS experiment. The first two case/control longitudinal sample sets were analyzed on an LTQ Orbitrap XL (Thermo Scientific, Waltham, Mass.) mass spectrometer and the third case/control sample set was analyzed on a Q Exactive Plus mass spectrometer (Thermo Scientific). Both instruments were equipped with Nano-Acquity (Waters, Milford, Mass.) pumps and a column heater maintained at 40° C. Tryptic digests were injected onto a UPLC Symmetry trap column (180 μm i.d.×2 cm packed with 5 μm C18 resin; Waters), and peptides were separated by RP-HPLC on a BEH C18 nanocapillary analytical column (75 μm i.d.×25 cm, 1.7 μm particle size, Waters) at a flow rate of 200 nL/min. Solvent A was Milli-Q (Millipore, Billerica, Mass.) water containing 0.1% formic acid, and Solvent B was acetonitrile containing 0.1% formic acid. The 85 minute gradient used for samples analyzed on the Oribitrap XL was as previously described in Beer et al., J. Proteome Res. 2011, 10, 1126-1138, while a slightly extended 95 minute gradient consisting of 5-30% B over 75 min, 30-80% B over 10 min, 80% B for 10 min, before returning to 5% B over 1 min was used for analysis of samples on the Q Exactive Plus. The HPLC, peak retention times, and mass spectrometer were carefully monitored throughout each experiment to ensure that performance was within tight tolerances in order to facilitate comparisons of LC-MS signals.

Raw mass spectrometric data were processed by MaxQuant software (Ver. 1.4.1.2) as previously described in Cox et al., Mol. Cell Proteomics 2014, 13, 2513-2526; Cox and Mann, Nat. Biotechnol. 2008, 26, 1367-1372. The “match between runs” option was enabled to match identifications across samples. The mass spectrometry proteomics data have been deposited to the ProteomeXchange Consortium (proteomecentral.proteomexchange.org) via the PRIDE partner repository with the dataset identifier PXD004058. Vizcaino et al., Nucleic Acids Res. 2013, 41, D1063-1069.

Peak lists were searched against the human Uniprot database with a full tryptic constraint using the Andromeda search engine. Carbamidomethyl cysteine was set as a fixed modification and methionine oxidation and N-terminus acetylation were set as variable modifications. Common expected contaminants including keratins and trypsin and a decoy database, produced by reversing the sequence of each protein, were appended to the forward database. Criteria for high confidence peptide/protein identifications included a false discovery rate (FDR) set to 1% for proteins and peptides, and removal of proteins identified by a single peptide. Relative abundance of each protein across all samples in an experiment was determined using the label-free quantitation option of MaxQuant. The software sums intensity of each full MS scan across all identified peptide peaks associated with a given protein. Protein identifications were filtered using Perseus software (perseus-framework.org) to remove reverse hits, contaminants, and proteins identified only by modified peptides or low confidence single peptide identifications. In addition, prior to statistical analysis, Perseus was used impute missing data points by creating a Gaussian distribution of random numbers to simulate the distribution of low signal values (imputation width=0.3, shift=1.8).

Due to different instruments and slightly different gradients used while analyzing cases/controls 1 and 2 (Orbitrap XL, 85 minute gradient) and case/control 3 (Q Exactive Plus, 95 minute gradient), the entire proteomes from all three case and control pairs in a single quantitative MaxQuant comparison were not analyzed. Hence, each case/control pair was analyzed individually and combined the results for interpretation and comparison. Specifically, identical protein groups across experiments were matched by their Uniprot accession numbers, and statistical tests, further described below, were performed on normalized and logarithmic intensities within each case and control pair. Heat maps of protein intensity z-scores and longitudinal trends were used to select the best markers which displayed increased or decreased protein levels between cases and controls.

### Example 6—Validation of Findings

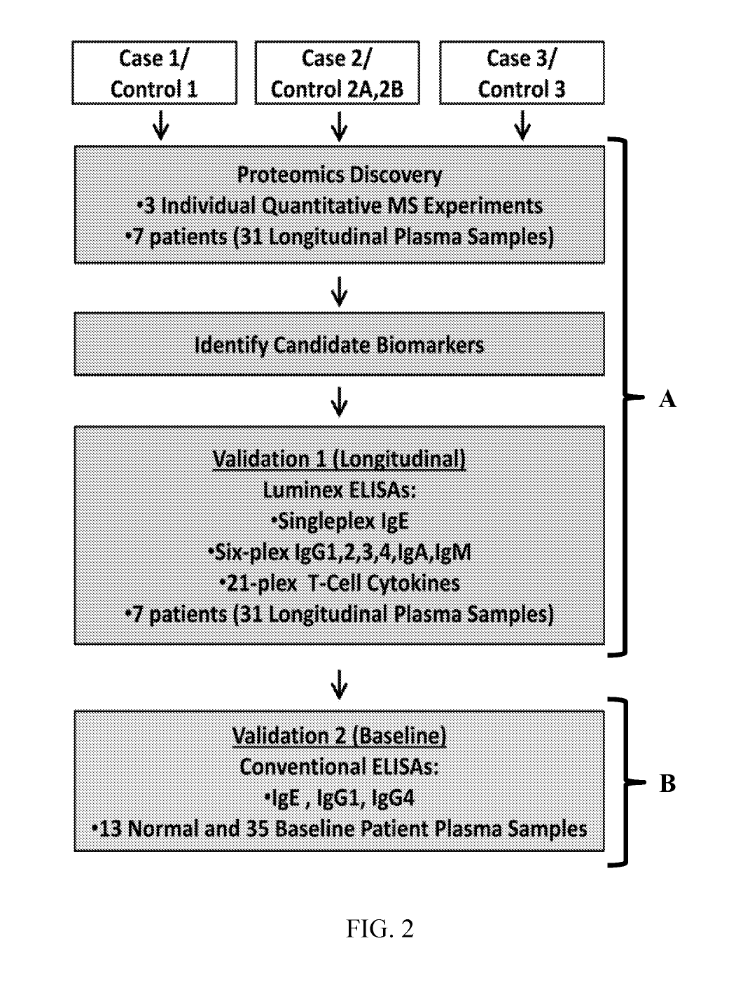

To verify the most promising proteomics discovery findings, singleplex IgE and multiplex IgG1, IgG2, IgG3, IgG4, IgA and IgM Isotyping assays were performed using Luminex kits (Millipore) according to the manufacturer's protocol (FIG. 2). The 31 longitudinal plasma samples from the 3 case/control pairs were diluted 1:50 or 1:16,000 for IgE and Isotyping kits, respectively, and added in duplicate on 96 well plates. Multiplex assays (Millipore) for Th1 and Th2 associated cytokines (IFN-γ, IL-4, IL-5, IL-6, IL-10, IL-13, GM-CSF, TNF-α, IL-2, IL-12, IL-113, IL-7, IL-8, IL-17A, IL-21, IL-23, MIP-1α, MIP-1β, MIP-3α, Fractalkine, ITAC) were also performed to further evaluate changes in IgE associated immune markers. Beads were read using a MAGPIX instrument (Millipore) and data were analyzed with Milliplex Analyst software (version 5.1).

In order to define the immunoglobulin levels amongst healthy participants, thirteen plasma EDTA samples were obtained from healthy female donors at the University of Pennsylvania (n=5) and the Wistar Institute (n=8). Participants were without evidence of comorbid conditions, including cardiovascular or oncologic disease. Plasma samples were processed and stored in an identical manner to the cancer cohort, and tested on the platforms as detailed above.

A standard colorimetric ELISA assay on baseline samples from the doxorubicin and trastuzumab cohort and normal healthy participants was obtained. Sandwich ELISA assays (Affymetrix eBioscience, San Diego, Calif.) were used to measure human IgE, IgG1 and IgG4 plasma levels at baseline (prior to treatment) for the entire cohort of 35 participants who received doxorubicin and trastuzumab, plus 13 normal female plasma donors (FIG. 2). ELISA plates were coated with each respective primary anti-human monoclonal antibodies and assays were performed according to manufacturer's instructions.

The high sensitivity T-cell 21-plex panel (Millipore) was also used to evaluate T-helper cell cytokines and chemokines (e.g. IFNγ, IL-4, IL-5, IL-6, IL-10, IL-13, GM-CSF, TNF-α, IL-2, IL-12, IL-1β, IL-7, IL-8, IL-17A, IL-21, IL-23, MIP-1α, MIP-1β, MIP-3α, Fractalkine, ITAC). 96-well plates were washed with 200 ul assay buffer. Next, 25 μl of the longitudinal plasma samples from the three case/control pairs were diluted 1:2 with assay buffer and added in duplicate to the plates. 25 μL of pre-mixed antibody-immobilized magnetic beads were added to each well followed by overnight incubation at 4° C. Plates were washed three times with 200 μl of wash buffer and 50 μl of detection antibodies were adder per well and incubated for 1 hr at RT. 50 μl of streptavidin-phycoerythrin was added to the wells and incubated for an additional 30 min. Finally, plates were washed three times with 200 μl wash buffer, 150 μL of sheath fluid was added to each well, and the resuspended beads were read on a MAGPIX instrument, as described above.

Sandwich ELISA assays (Affymetrix eBioscience, San Diego, Calif.) were used to measure human IgE, IgG1 and IgG4 plasma levels at baseline (prior to treatment) for the entire cohort of 35 patients who received doxorubicin and trastuzumab, plus 13 normal female plasma donors (FIG. 2). 96-well ELISA plates were coated with each respective primary anti-human monoclonal antibodies diluted in PBS and incubated overnight at 4° C. followed by overnight blocking with PBS containing 0.5% Tween 20 and 5% BSA. Plates were washed twice with PBS, 0.05% Tween 20 after blocking and each subsequent step. Plasma samples were diluted 1:10 (IgE), 1:2000 (IgG1), or 1:1000 (IgG4) in PBS containing 0.1% Tween 20 and 1% BSA and were added in triplicate to the plates and incubated for 2 hrs at RT. Horseradish peroxidase (HRP)-conjugated secondary anti-human monoclonal antibody was added to plates and incubated for 1 hr at RT. The plates were then developed by adding tetra 3,30,5,50-tetramethylbenzidine (TMB) substrate and incubated for 20 min at RT. Finally, the reaction was stopped by adding 2N sulfuric acid and plates were read at 450 nm.

Statistical methods were performed as follows. In two separate discovery analyses, diagnostic and predictive biomarkers were identified. Diagnostic biomarkers were defined as those proteins exhibiting a significant change in protein level at the same time as the onset of CTRCD. Candidate diagnostic biomarkers were selected by initially considering rate of change for individual cases; specifically, the level of a given protein was significantly higher or lower (two-tailed Student's t-test p-value <0.05 and fold change >1.5) at one or more pre-CTRCD timepoints compared with timepoints after diagnosis of CTRCD.

Predictive biomarkers were defined as those that exhibited overall significant differences between case and control at baseline or at all timepoints; that is, there was a consistent difference between case and control starting at baseline and persisting throughout the study. Significantly changed proteins were defined as having both >1.5 fold change between the average of all timepoints for each case and control pair, and a Student t-test p-value <0.05.

For Luminex validation studies, differences between case and control samples were calculated at baseline using a two-tailed Student's t-test. For the ELISA data derived from the 35 baseline samples, the non-parametric Wilcoxon Rank Sum test was used to compare baseline IgE levels from individuals in the doxorubicin/trastuzumab cohort who developed CTRCD with those individuals who did not develop CTRCD. Logistic regression models were then used to determine the associations between baseline levels of biomarkers and odds of CTRCD. Baseline IgE levels and IgE/IgG1 ratios were transformed on the log₂ scale for these analyses. Area under the receiver operating characteristic curves (AUC) were calculated to assess the discriminative ability of each biomarker. The biomarker cutpoint at which the optimal sensitivity and specificity could be achieved was also calculated. Statistical significance was set at p<0.05 for all analyses.

### Example 7—Results for Patient Characteristics of Discovery Cohort

Cases and controls selected for the proteomics discovery analyses had the following characteristics, as detailed in Table 2. All participants received a regimen containing doxorubicin (240 mg/m²), cyclophosphamide, followed by paclitaxel and trastuzumab for 4 cycles, followed by 1 year of trastuzumab therapy. All participants also received radiotherapy. No participants had any history of cardiovascular disease or risk factors prior to cancer therapy.

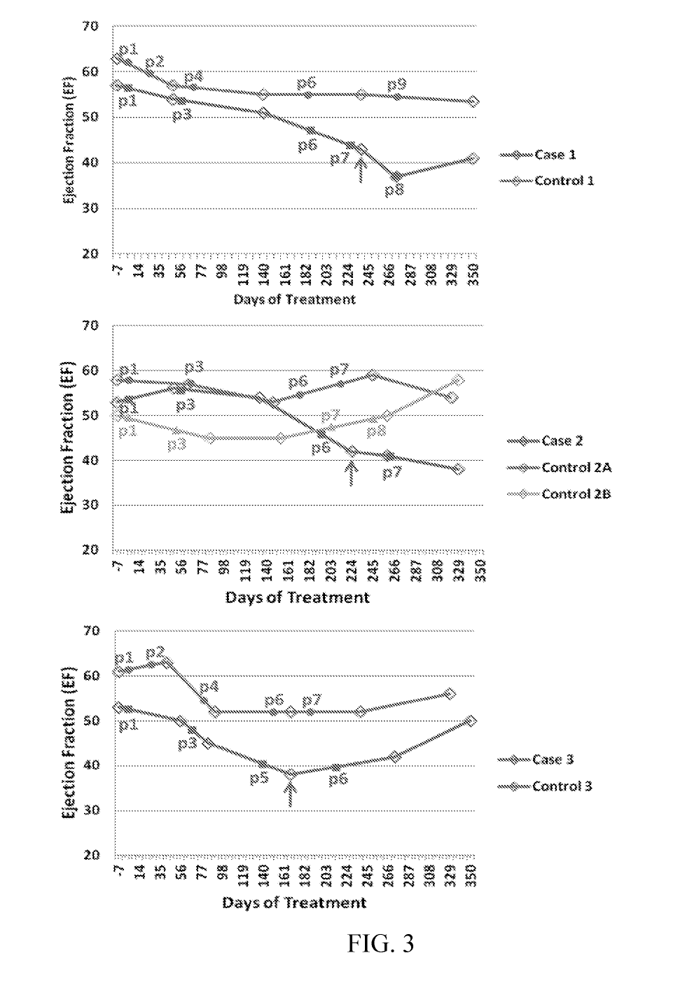

In the cases, CTRCD occurred between 167 to 238 days. Moreover, cases all complained of heart failure symptoms including dyspnea on exertion and fatigue, and were all started on cardiac medications after diagnosis, including angiotensin converting enzyme-inhibitors (ACE-I) and beta blockers. Controls did not have any evidence of significant or sustained declines in EF (i.e., CTRCD) or symptoms of heart failure. Timeline plots of clinical assessment of cardiac function by EF and the relationship to analyzed plasma fractions are shown in FIG. 3.

### Example 8—Results for Proteomics Biomarker Discovery

As previously noted, case/control pairs 1 and 2 were analyzed using an Orbitrap XL mass spectrometer, while case/control 3 was analyzed on a Q Exactive Plus instrument when this newer, higher performance instrument became available. Approximately 862 proteins were identified from case/control 1 and 2 plasma proteomes while analysis of case/control 3 resulted in the identification of 1,360 proteins. The increased depth of analysis achieved in case/control 3 was seen as potentially valuable, because most of the additional ˜500 proteins identified in this dataset should be very low abundance proteins that were below the detection limit of the Orbitrap XL mass spectrometer.

Diagnostic biomarkers were expected to be those proteins that showed an increase or decrease in protein abundance specifically associated with the timeframe of CTRCD development. Surprisingly, while a number of proteins exhibited large changes in abundance over time, none of these protein changes were significantly associated with onset of CTRCD.

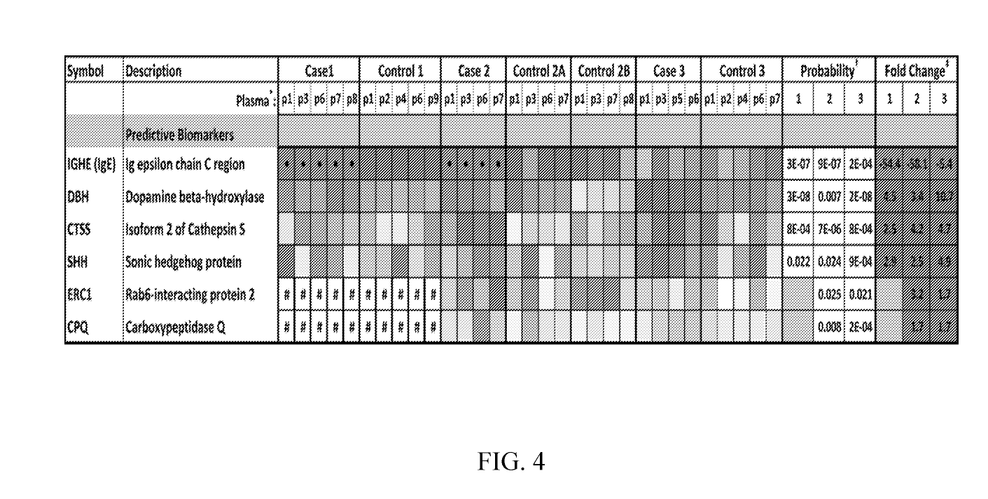

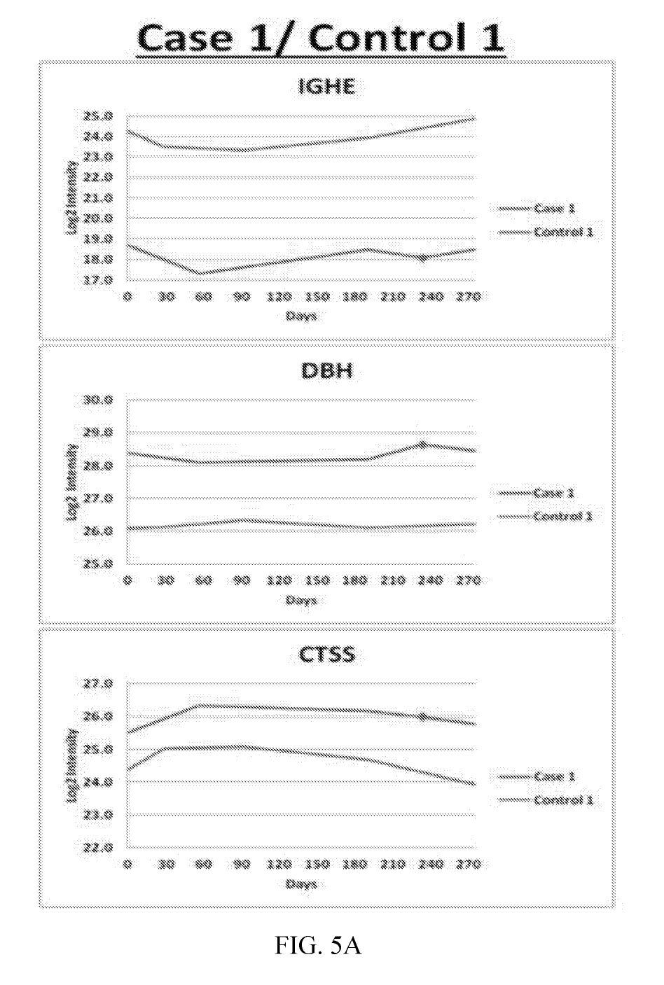

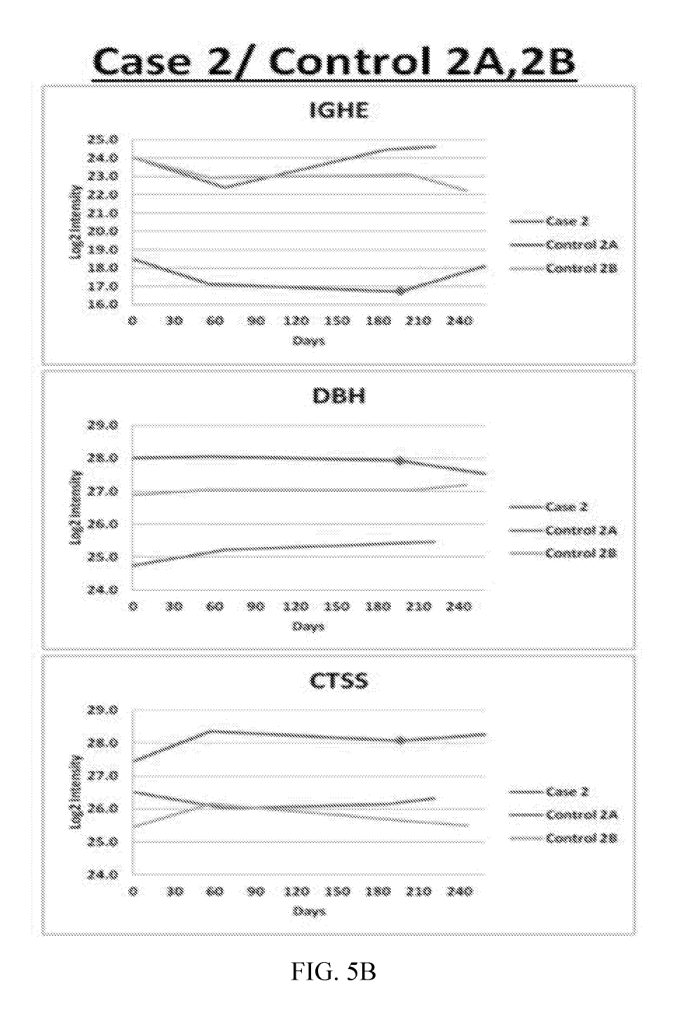

Predictive biomarkers were proteins which exhibited differences in the level between case and control either prior to treatment (baseline, at the time of first plasma collection) or at all timepoints for cases and controls (greater than 1.5-fold change between averages and p<0.05). The six best scoring candidate predictive biomarkers are summarized in the heatmap of FIG. 4. Longitudinal trends were also evaluated for each patient analyzed in the discovery experiments. Interestingly, the three proteins with the largest overall case/control differences (FIGS. 5A to 5C) were either consistently lower in all case timepoints as compared with matched controls, i.e., immunoglubulin E (IgE), or higher in cases as compared with matched controls, i.e., dopamine beta-hydroxylase (DBH) and cathepsin S (CTSS). Baseline immunoglobulin E (IgE) was a focus for validation because it showed the largest differences, from 5- to 58-fold, between cases and controls (FIG. 4).

### Example 9—Results for Validation of Baseline IgE as a Biomarker of Cardiac Dysfunction

Our most promising biomarker, IgE, was identified in the proteome comparisons based on the epsilon chain C region of immunoglobulin. IgE is the lowest abundance immunoglobulin in plasma, with concentrations in the low ng/ml range. Prabhu et al., Ann. Allergy Asthma Immunol. 1997, 78, 45-53. Importantly, unlike other immunoglobulins, IgE was not a target of the antibody column used to deplete abundant proteins. Both the heat maps and the longitudinal trend plots show that this protein was consistently low at all timepoints in all three cases in the discovery proteomics analyses (FIG. 4 and FIGS. 5A to 5C).

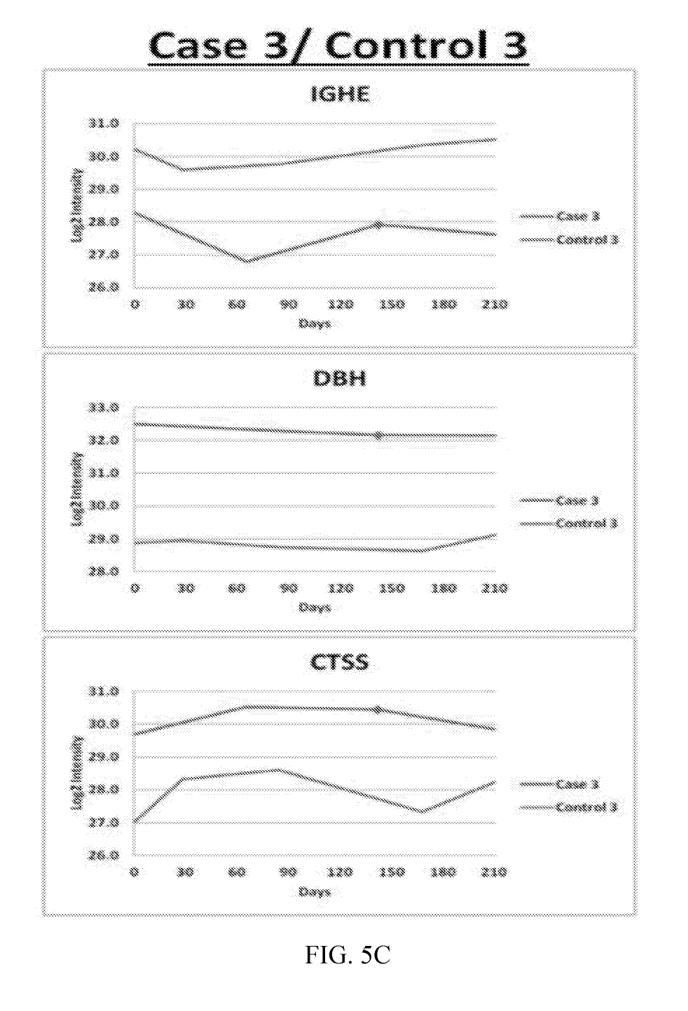

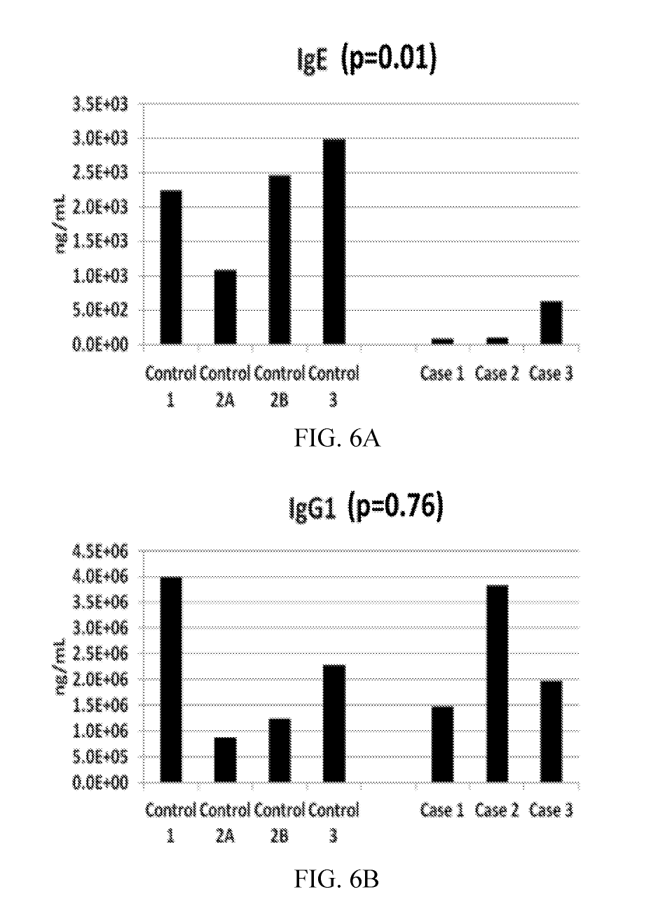

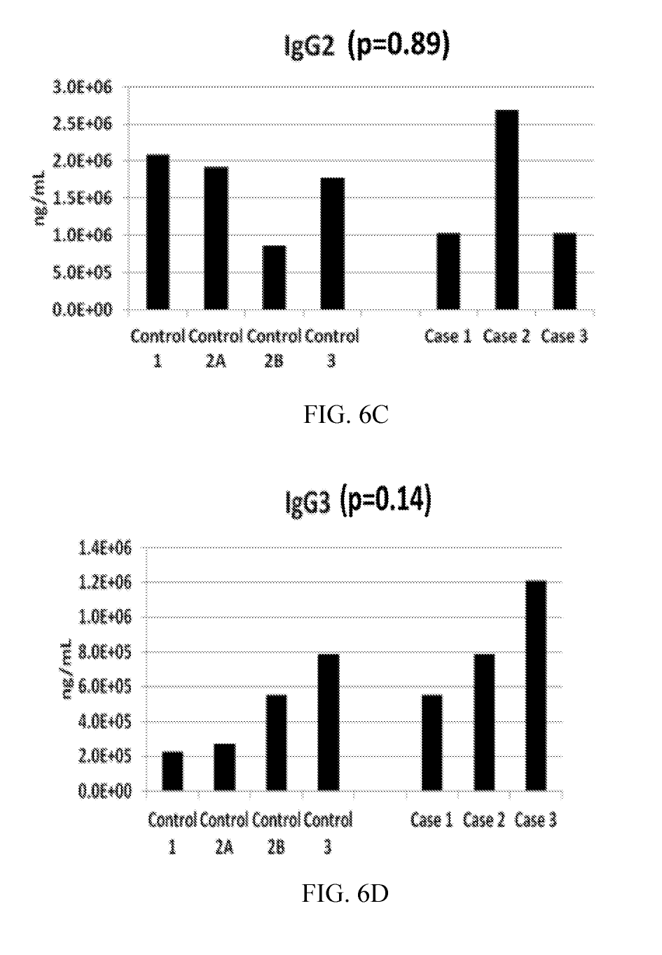

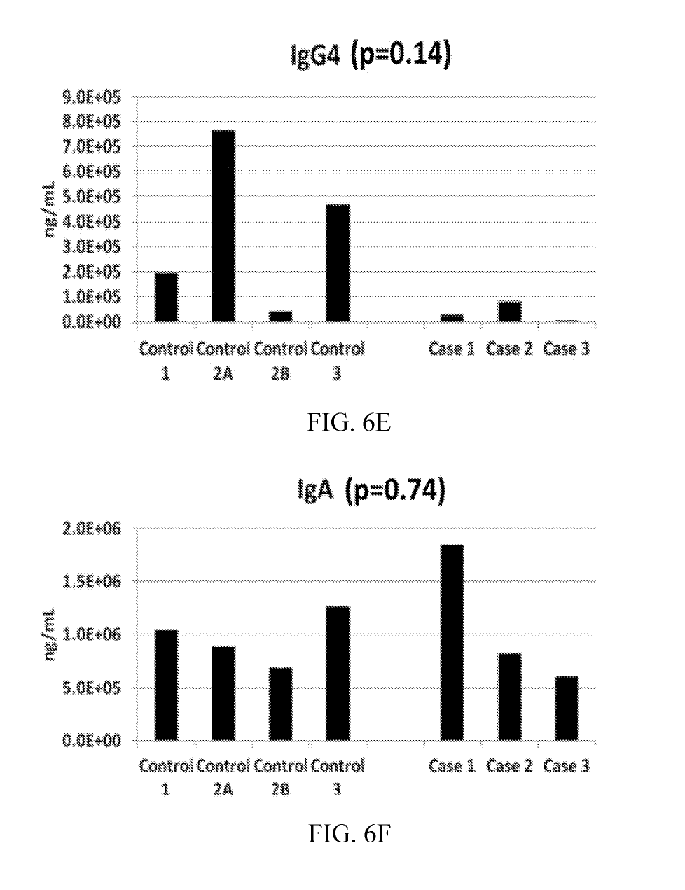

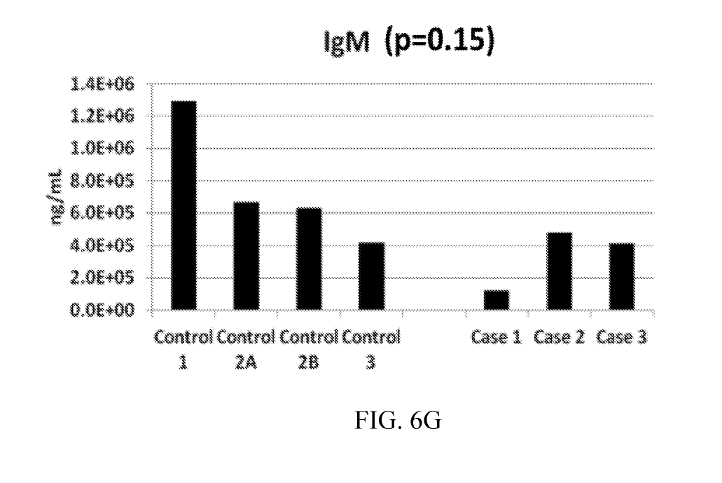

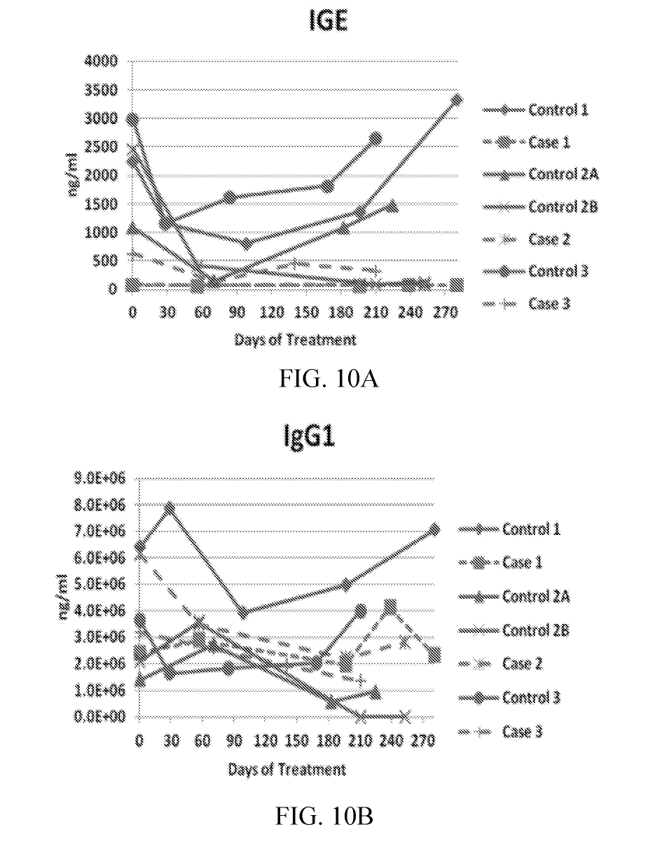

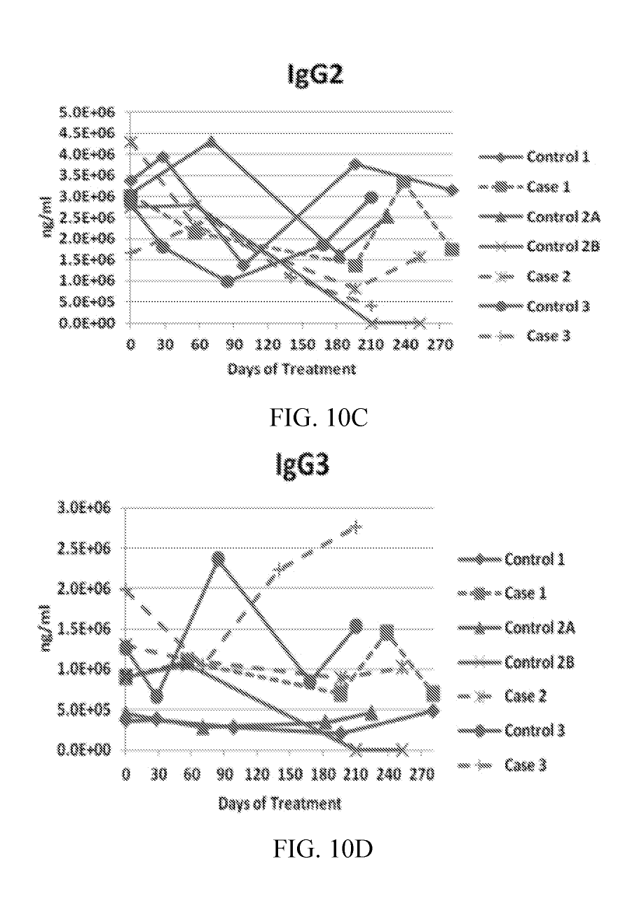

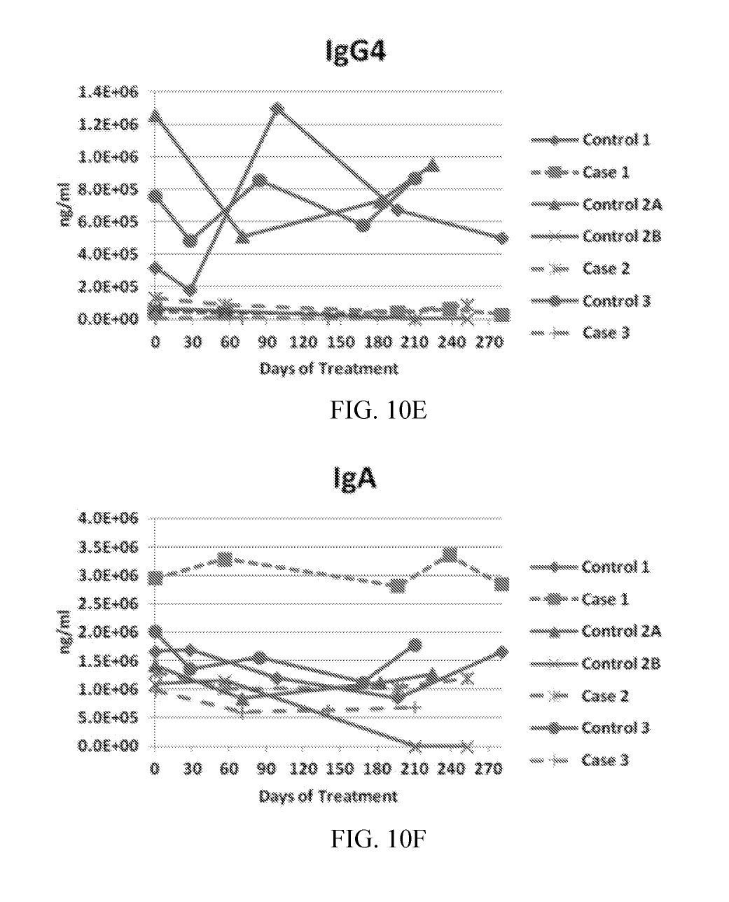

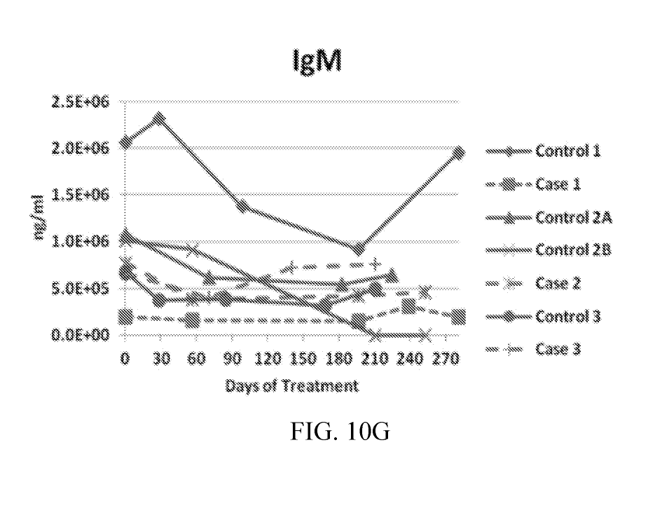

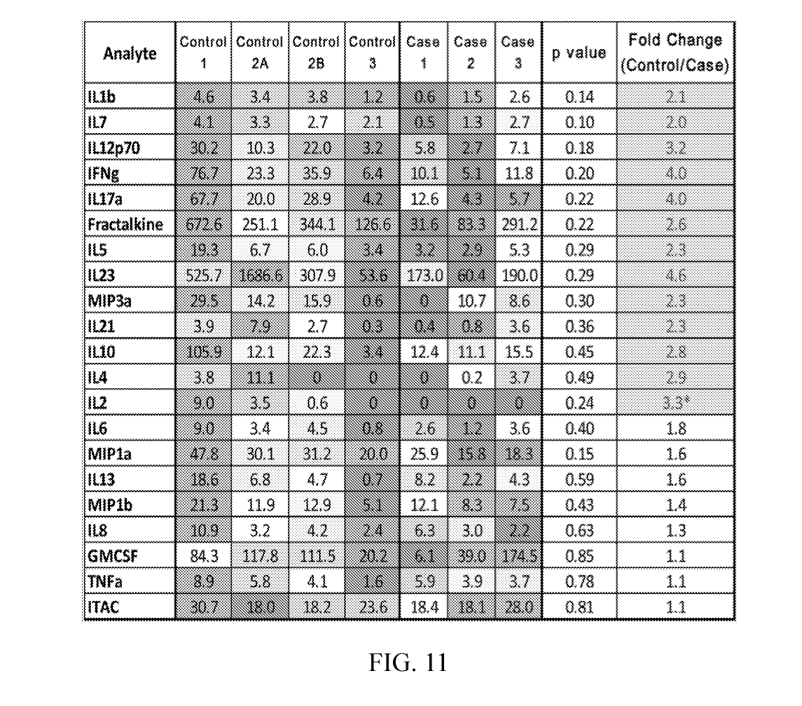

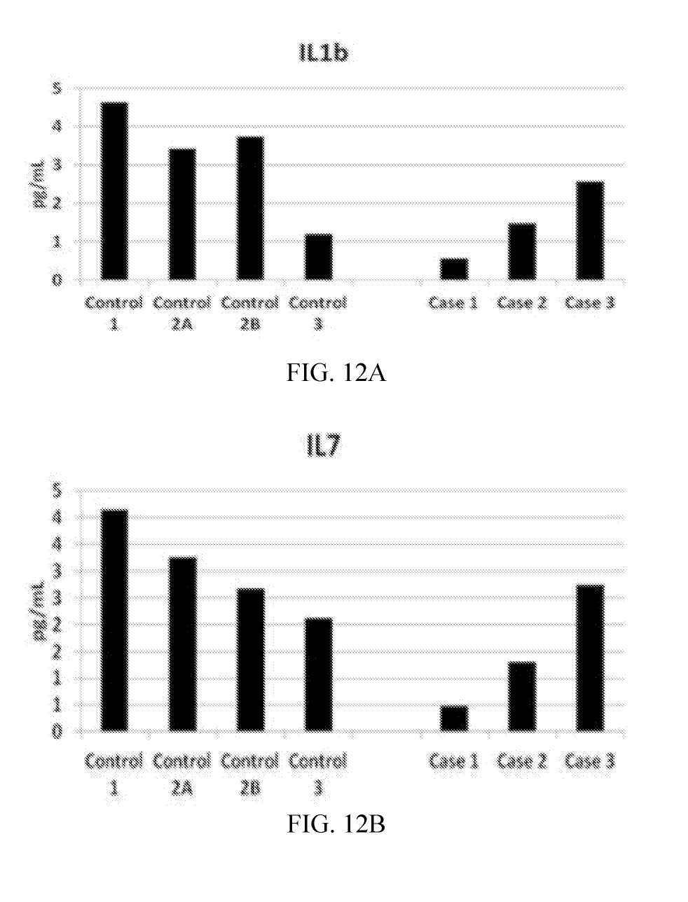

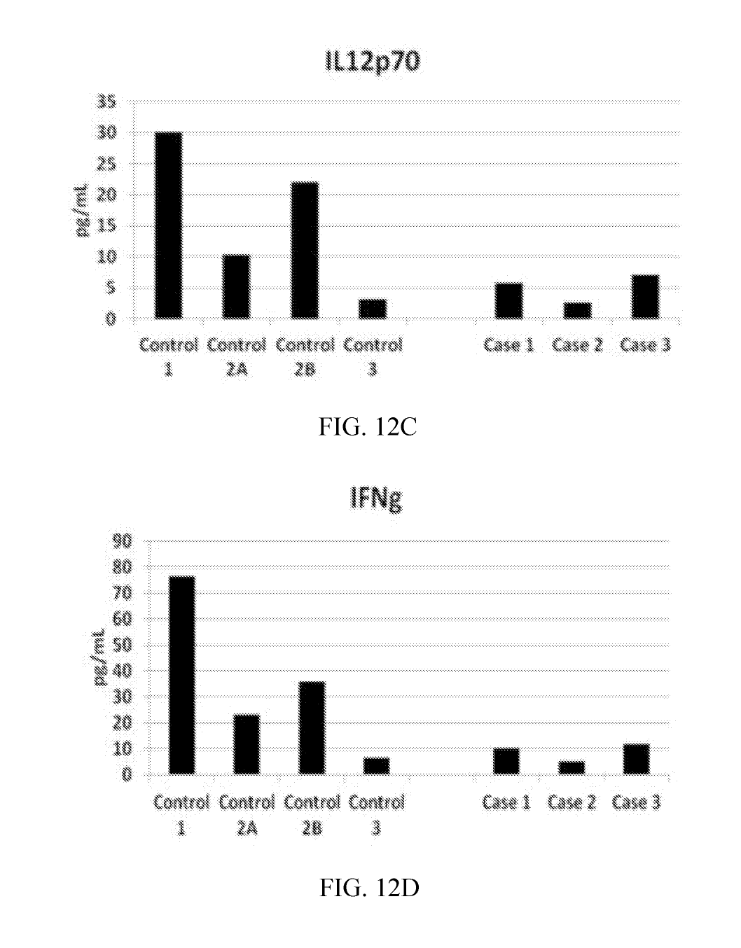

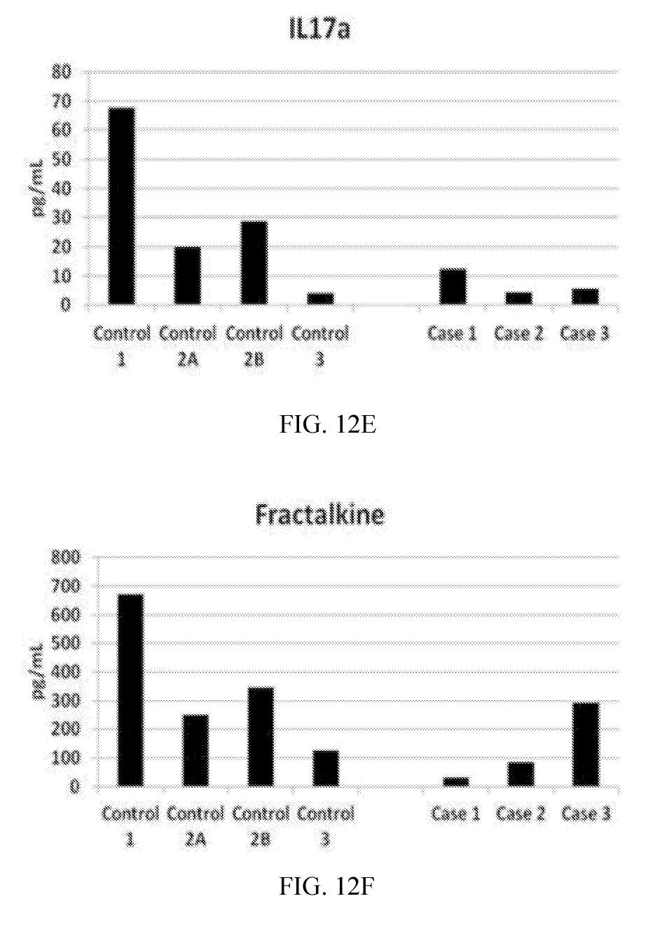

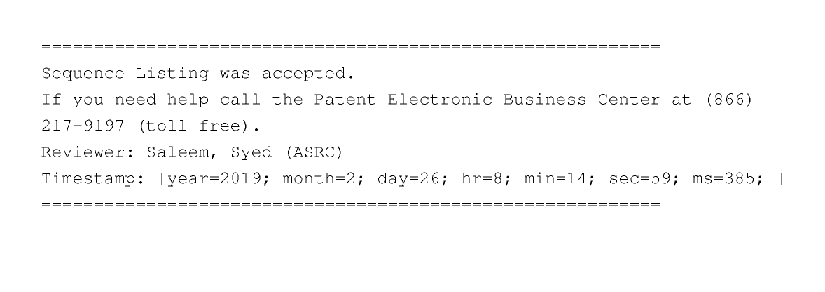

To verify the discovery results for IgE and to determine whether the observed lower IgE levels in cases were indicative of a general suppression of the immune system, all immunoglobulin subtype levels were quantitated using a multiplexed Luminex assay platform in the 3 cases and 4 matched controls. These results are displayed graphically in FIGS. 6A to 6G which details baseline immunoglobulin data only and in FIGS. 10B to 10G which includes data from all timepoints. There was no evidence of a general immunosuppression in cases either at baseline (FIGS. 6B to 6G) or throughout therapy in the longitudinal plasma samples (FIGS. 10B to 10G). Importantly, the IgE differences at baseline (FIG. 6A) and the longitudinal trends observed in the proteome discovery experiments (FIG. 10A) were verified using this independent Luminex assay. When baseline levels in cases and controls were compared, there was a highly significant difference between groups with controls being higher (p=0.01). An additional interesting observation that arose from these initial validation experiments is that IgG4 levels were also low at baseline for all cases (FIGS. 6B to 6G). To further investigate the role of the immune system in doxorubicin and trastuzumab CTRCD, IgE related Th1 and Th2 cytokine profiles were evaluated for cases and controls. Interestingly, a number of IgE related cytokines such as IL4, IL5, IL17, and fractalkine were also elevated in controls as compared to cases at baseline (FIG. 11 and FIGS. 12A to 12F). Chong et al., Ann Clin Lab Sci. 2016, 46, 168-173; Milovanovic et al., J. Invest. Dermatol. 2010, 130, 2621-2628; Reubsaet et al., Allergy 2014, 69, 406-410.

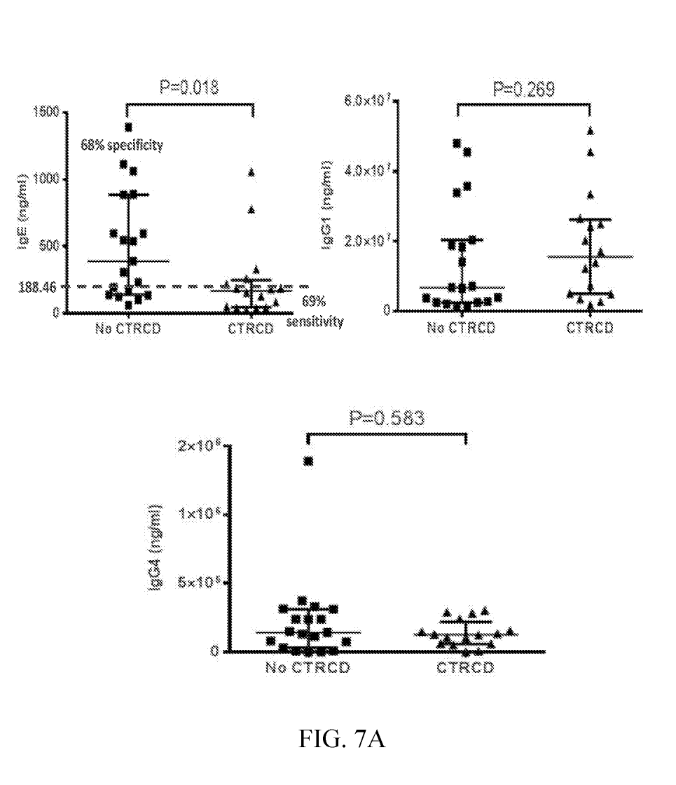

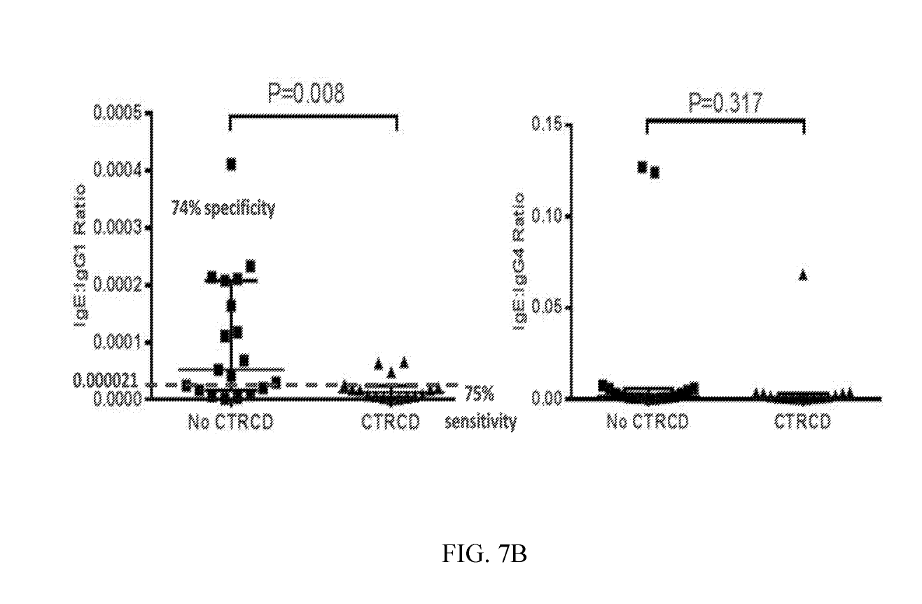

Based on these results, a more comprehensive evaluation of IgE, IgG1, and IgG4 levels was performed by assaying the baseline plasma samples of all thirty-five participants in the doxorubicin/trastuzumab cohort (Table 3). Conventional colorimetric ELISA assays were used for this second set of validation experiments. The results demonstrated that baseline IgE levels were significantly higher (p=0.018) in participants who did not experience CTRCD [mean 498.8 ng/ml±401.0; median 389.3 ng/mL with range 60.5-1392.1] compared to those who suffered from CTRCD [mean 234.9 ng/mL±285.9; median 167 ng/mL with range 23.2-1059.2], suggesting a potential protective role for elevated IgE at baseline in cancer patients undergoing cardiotoxic therapy (FIG. 7A). In this cohort, 9 of the thirty-five participants had a history of allergies or asthma, or were taking allergy medications. However, there was no relationship between allergy and baseline IgE levels, and the distribution of participants with a history of allergies or asthma was similar across participants with and without CTRCD.

TABLE 3
Clinical Characteristics of Validation Cohort (N = 35).

| Clinical Characteristics | N (%) or Median (IQR) | |
|---|---|---|
| Age, years | 45 | (39, 57) |
| Race | | |
| Caucasian | 21 | (60) |
| Black | 10 | (29) |
| Other or unknown | 4 | (11) |
| Breast cancer side | | |
| Left | 16 | (46) |
| Right | 14 | (40) |
| Bilateral | 5 | (14) |
| Breast cancer nodal status | | |
| N0 | 15 | (43) |
| N1 | 13 | (37) |
| N2 | 5 | (14) |
| N3 | 2 | (6) |
| Breast cancer hormonal status | | |
| ER+ | 18 | (51) |
| Left-sided Radiotherapy | 16 | (46) |
| Baseline EF, % | 55 | (54, 59) |
| Cancer Therapeutics Related Cardiac | 18 | (51) |
| Dysfunction, % | | |

Samples from a cohort of healthy female volunteers without asthma or allergies and without evidence of any comorbidity including cardiovascular disease or cancer was analyzed to determine IgE levels in normal individuals [mean 179.6 ng/ml±231.7; median 97.0 ng/ml (range 36.9-853.8)]. This comparison did not reveal any significant difference between cases who developed CTRCD with normal individuals (p=0.99). In contrast, controls who did not experience CTRCD were significantly higher in comparison to non-cancer, healthy female volunteers (p=0.007). These results suggest that cancer patients who did not experience CTRCD had elevated IgE levels at baseline that may offer a cardioprotective benefit.

IgG1 and IgG4 levels were not significantly different between individuals with or without CTRCD, nor was the ratio between IgE/IgG4 (p>0.05 for all) (FIG. 7B). However, when the ratio of IgE/IgG1 was evaluated, the differences in patients who experienced CTRCD compared to those who did not were most pronounced (p=0.008). Similarly, logistic regression models of IgE and the IgE/IgG1 ratio demonstrated a significant association with high IgE levels and a decreased probability of cardiac dysfunction. Each doubling of IgE was associated with an odds ratio (OR) of 0.52 (95% CI 0.31, 0.90, p=0.018) for the development of CTRCD. For the ratio of IgE/IgG1, each doubling was associated with an OR of 0.63 (95% CI 0.43, 0.92 p=0.017). Moreover, area under the receiver operating characteristics curve (AUC) analyses suggest strong discriminative ability with IgE and IgE/IgG1 with AUCs of 0.73 and 0.76, respectively (FIG. 8). The sensitivities and specificities at various cutpoints of IgE and IgE/IgG1 were also evaluated. For IgE, a measure of 188.5 ng/ml had a combined sensitivity of 69% and specificity of 68%. The IgE/IgG1 ratio at a cut-point of 2.05×10⁻⁵ demonstrated the highest combined sensitivity of 75% and specificity of 74%. Overall, these findings indicate that elevated IgE or IgE/IgG1 levels prior to doxorubicin and trastuzumab therapy may have a protective effect against cardiac dysfunction in patients undergoing this therapy.

### Example 10—Discussion of Results

In this study, state-of-the-art proteomic profiling techniques to uncover potential new biomarkers for doxorubicin and trastuzumab CTRCD were utilized. Using a carefully phenotyped cohort of breast cancer patients, it was discovered that differences in baseline circulating IgE levels between patients who suffer from doxorubicin and trastuzumab-induced cardiac dysfunction compared to those who do not. These novel findings present a new paradigm in CTRCD, and implicate a specific, under-studied component of the immune system as a potential mediator of doxorubicin and trastuzumab-induced cardiac dysfunction.

For the specific application of assessing risk of cardiac dysfunction in response to breast cancer therapies, the current results indicate that IgE and IgG1 levels could be measured prior to initiation of the therapeutic regimen as a method for stratifying patients for cardiac dysfunction risk. Those participants with low IgE and low IgE/IgG1 ratios appear to be at higher risk of developing cardiac dysfunction and might be more closely monitored for development of cardiac dysfunction.

A number of patent and non-patent publications may be cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

1. A method of treating a cancer in a human subject wherein the human subject exhibits an elevated concentration of immunoglobulin E (IgE) in plasma obtained from the human subject, comprising the step of administering a therapeutically effective amount of a chemotherapeutic regimen to the human subject in need thereof.

2. The method of claim 1, wherein the chemotherapeutic regimen is selected from the group consisting of: doxorubicin monotherapy; trastuzumab monotherapy; doxorubicin and trastuzumab combination therapy; doxorubicin, cyclophosphamide, and 5-fluorouracil combination therapy; and doxorubicin, cyclophosphamide, paclitaxel, and trastuzumab combination therapy.

3. The method of claim 1, wherein the cancer is breast cancer.

4. The method of claim 1, wherein the human subject further exhibits a reduced concentration of beta-hydroxylase in plasma obtained from the human subject.

5. The method of claim 1, wherein the human subject further exhibits a reduced concentration of cathepsin S in plasma obtained from the human subject.

6. The method of claim 1, wherein the elevated concentration of IgE is determined by an IgE-specific protein assay.

7. The method of claim 6, wherein the IgE-specific protein assay is an enzyme-linked immunosorbent assay (ELISA).

8. The method of claim 6, wherein the IgE-specific protein assay is liquid chromatography mass spectrometry (LC-MS) assay.

9. The method of claim 1, wherein the elevated concentration of IgE is determined as a measurement of IgE concentration in plasma, wherein the IgE concentration in plasma is selected from the group consisting of greater than 150 ng/mL, greater than 200 ng/mL, greater than 300 ng/mL, and greater than 400 ng/mL.

10. The method of claim 1, wherein the elevated concentration of IgE is determined as a measurement of IgE relative to immunoglobulin G1 (IgG1) in plasma (IgE/IgG1 ratio), wherein IgE/IgG1 ratio is selected from the group consisting of greater than 1.5×10⁻⁵, greater than 2×10⁻⁵, greater than 2.5×10⁻⁵, greater than 3×10⁻⁵, greater than 4×10⁻⁵, and greater than 5×10⁻⁵.

11. A method of treating a cancer in a human subject comprising the steps of:
(a) obtaining a plasma sample from the human subject;
(b) analyzing the plasma sample by an immunoglobulin E (IgE)-specific protein assay for IgE;
(c) determining whether the human subject is at a low risk for cardiac injury from a chemotherapeutic regimen based on an elevated IgE concentration; and
(d) administering a chemotherapeutic regimen to the human subject determined to have the low risk of cardiac injury.

12. The method of claim 11, wherein the chemotherapeutic regimen is selected from the group consisting of: doxorubicin monotherapy; trastuzumab monotherapy; doxorubicin and trastuzumab combination therapy; doxorubicin, cyclophosphamide, and 5-fluorouracil combination therapy; and doxorubicin, cyclophosphamide, paclitaxel, and trastuzumab combination therapy.

13. The method of claim 11, wherein the cancer is breast cancer.

14. The method of claim 11, further comprising the steps of analyzing the plasma sample for beta-hydroxylase and determining the risk of cardiac injury in the human subject based on reduced beta-hydroxylase concentration.

15. The method of claim 11, further comprising the step of analyzing the plasma sample for cathepsin S and determining the risk of cardiac injury in the human subject based on reduced cathepsin S concentration.

16. The method of claim 11, wherein the IgE-specific protein assay is an enzyme-linked immunosorbent assay (ELISA).

17. The method of claim 11, wherein the IgE-specific protein assay is liquid chromatography mass spectrometry (LC-MS) assay.

18. The method of claim 11, wherein the elevated IgE concentration is determined as a measurement of IgE concentration in plasma, wherein the elevated IgE concentration in plasma is selected from the group consisting of greater than 150 ng/mL, greater than 200 ng/mL, greater than 300 ng/mL, and greater than 400 ng/mL.

19. The method of claim 11, wherein the elevated IgE concentration is determined as a measurement of IgE relative to immunoglobulin G1 (IgG1) in plasma (IgE/IgG1 ratio), wherein the IgE/IgG1 ratio is selected from the group consisting of greater than 1.5×10⁻⁵, greater than 2×10⁻⁵, greater than 2.5×10⁻⁵, greater than 3×10⁻⁵, greater than 4×10⁻⁵, and greater than 5×10⁻⁵.

20. A method of preventing injury in a human subject being treated for a cancer comprising the steps of:
(a) obtaining a plasma sample from the human subject;
(b) analyzing the plasma sample by an immunoglobulin E (IgE)-specific protein assay for IgE;
(c) determining whether the human subject is at a high risk for cardiac injury from a chemotherapeutic regimen based on a reduced IgE concentration; and
(d) preventing administration of a chemotherapeutic regimen to the human subject determined to have the high risk of cardiac injury.

21. The method of claim 20, wherein the chemotherapeutic regimen is selected from the group consisting of: doxorubicin monotherapy; trastuzumab monotherapy; doxorubicin and trastuzumab combination therapy; doxorubicin, cyclophosphamide, and 5-fluorouracil combination therapy; and doxorubicin, cyclophosphamide, paclitaxel, and trastuzumab combination therapy.

22. The method of claim 20, wherein the cancer is breast cancer.

23. The method of claim 20, further comprising the steps of analyzing the plasma sample for beta-hydroxylase and determining the risk of cardiac injury in the human subject based on elevated beta-hydroxylase concentration.

24. The method of claim 20, further comprising the step of analyzing the plasma sample for cathepsin S and determining the risk of cardiac injury in the human subject based on elevated cathepsin S concentration.

25. The method of claim 20, wherein the IgE-specific protein assay is an enzyme-linked immunosorbent assay (ELISA).

26. The method of claim 20, wherein the IgE-specific protein assay is liquid chromatography mass spectrometry (LC-MS) assay.

27. The method of claim 20, wherein the reduced IgE concentration is determined as a measurement of IgE concentration in plasma, wherein the reduced IgE concentration in plasma is selected from the group consisting of less than 100 ng/mL, less than 150 ng/mL, less than 200 ng/mL, and less than 250 ng/mL.

28. The method of claim 20, wherein the reduced IgE concentration is determined as a measurement of IgE relative to immunoglobulin G1 (IgG1) in plasma (IgE/IgG1 ratio), wherein the IgE/IgG1 ratio is selected from the group consisting of less than 1.5×10⁻⁵, less than 2×10⁻⁵, less than 2.5×10⁻⁵, less than 3×10⁻⁵, less than 4×10⁻⁵, and less than 5×10⁻⁵.

29. A kit for determining the risk of cardiac injury in a human subject receiving chemotherapy, comprising an assay for determining the concentration of immunoglobulin E (IgE) in plasma obtained from the human subject.

30. The kit of claim 29, wherein the assay for determining the concentration of IgE is an enzyme-linked immunosorbent assay (ELISA).

31. The kit of claim 29, further comprising an assay for determining the concentration of immunoglobulin G1 (IgG1).

32. The kit of claim 31, wherein the assay for determining the concentration of IgG1 is an enzyme-linked immunosorbent assay (ELISA).

33. The kit of claim 29, further comprising an assay for determining the concentration of beta-hydroxylase.

34. The kit of claim 29, further comprising an assay for determining the concentration of cathepsin S.


SEQUENCE LISTING (10 sequences)

SEQ ID NO: 1 - protein, 451 aa, Artificial Sequence
  trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 117 aa, Artificial Sequence
  trastuzumab variable heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVT

SEQ ID NO: 4 - protein, 103 aa, Artificial Sequence
  trastuzumab variable light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTK

SEQ ID NO: 5 - protein, 10 aa, Artificial Sequence
  trastuzumab variable heavy chain CDR1
GFNIKDTYIH

SEQ ID NO: 6 - protein, 17 aa, Artificial Sequence
  trastuzumab variable heavy chain CDR2
RIYPTNGYTRYADSVKG

SEQ ID NO: 7 - protein, 11 aa, Artificial Sequence
  trastuzumab variable heavy chain CDR3
WGGDGFYAMDY

SEQ ID NO: 8 - protein, 11 aa, Artificial Sequence
  trastuzumab variable light chain CDR1
RASQDVNTAVA

SEQ ID NO: 9 - protein, 7 aa, Artificial Sequence
  trastuzumab variable light chain CDR2
SASFLYS

SEQ ID NO: 10 - protein, 9 aa, Artificial Sequence
  trastuzumab variable light chain CDR3
QQHYTTPPT